(19) United States
(12) Patent Application Publication
(10) Pub. No.: US 2025/0268993 A1
(43) Pub. Date: Aug. 28, 2025

(54) ALKALINE PHOSPHATE-BASED ONCOLOGY TREATMENTS

(71) Applicant: Theriva Biologics, Inc., Rockville, MD (US)

(72) Inventors: Christian FURLAN FREGUIA, Rockville, MD (US); Michael KALEKO, Rockville, MD (US)

(21) Appl. No.: 19/196,290

(22) Filed: May 1, 2025

Related U.S. Application Data
(63) Continuation of application No. 17/608,252, filed on Nov. 2, 2021, now Pat. No. 12,318,434.
(60) Provisional application No. 62/843,850, filed on May 6, 2019.

(51) Int. Cl. A61K 38/46; A61K 31/513; A61P 35/00

(52) U.S. Cl. CPC A61K 38/465; A61K 31/513; A61P 35/00; C12Y 301/03001

(57) ABSTRACT

The present invention relates to methods and compositions for preventing, treating, or reducing chemotherapy treatment-mediated side effects, including gastrointestinal (GI) side effects, comprising administering to a subject intestinal alkaline phosphatases (IAP).

### CROSS-REFERENCE TO RELATED APPLICATIONS

This application claims the benefit of U.S. Provisional Application No. 62/843,850, filed May 6, 2019, the entire contents of which are hereby incorporated by reference in their entirety.

### TECHNICAL FIELD

The present invention relates, inter alia, to methods for prevention and/or reduction of chemotherapy-mediated gastrointestinal side effects by administering therapeutic intestinal alkaline phosphatases.

### DESCRIPTION OF THE TEXT FILE SUBMITTED ELECTRONICALLY

The content of the text file submitted electronically herewith is incorporated herein by reference in their entirety: A computer readable format copy of the Sequence Listing (Filename: “SYN-046PC_ST25.txt”; Date created: May 4, 2020; File size: 84,284 bytes).

### BACKGROUND

Chemotherapy is the common approach to cancer treatment that has been widely used, and various chemotherapeutic agents have been developed for cancer treatment. However, many chemotherapeutic agents have side effects, including moderate to severe side effects, which can include gastrointestinal (GI) distress such as, for example, diarrhea and/or colitis. Some chemotherapeutic agents have been linked to dysbiosis in the intestinal microbiome, which is one of the main causes of intestinal mucositis. The side effects limit the application of chemotherapeutic agents.

Given the need for improved cancer therapies and mitigation of harmful side effects to allow for improved cancer patient care, there is a growing need for therapeutic compositions that prevent and/or reduce the side effects associated with administration of various anticancer agents.

Alkaline phosphatase (“AP,” EC 3.1.3.1) is a hydrolase enzyme that can remove phosphate groups from various targets including nucleotides and proteins. Alkaline phosphatases are found in prokaryotic as well as eukaryotic organisms ranging from E. coli to mammals. In particular, mammalian APs have been shown to play important roles in gut homeostasis, mucosal barrier function, promotion of commensal bacteria, and defense from pathogens. Mammalian APs exert their properties by primarily targeting LPS (a TLR4 agonist), flagellin (a TLR5 agonist) and CpG DNA (a TLR9 agonist). APs also degrade intestine luminal NTPs (e.g., ATP, GTP, etc.), which promote the growth of good bacteria and reverses dysbiosis. Accordingly, APs may find clinical use in, for example, preventing and/or reducing the side effects associated with administration of anticancer agents.

### SUMMARY

Accordingly, the present invention provides methods for using an intestinal alkaline phosphatase (IAP), along with a chemotherapeutic treatment, for treating and/or preventing side effects of the chemotherapeutic treatment.

In some aspects, the present invention provides a method for enhancing the therapeutic efficacy of a chemotherapeutic treatment comprising a nucleoside analog in a subject in need thereof that includes administering to the subject an effective amount of an IAP.

In some aspects of the present invention, a method for treating or preventing at least one side effect of a chemotherapeutic treatment with a nucleoside analog in a subject is provided, comprising administering to the subject an intestinal alkaline phosphatase (IAP), wherein the subject has undergone or is undergoing a treatment of a cancer using the chemotherapeutic treatment.

The IAP can be administered sequentially, simultaneously, or intermittently with the administration of the nucleoside analog. The IAP can be administered orally. In some embodiments, the nucleoside analog can is a purine or pyrimidine analog or derivatives thereof. Non-limiting examples of the nucleoside analog include 5-fluorouracil, 5′-deoxyfluorouridine, 5-fluoro-2′-deoxyuridine triphosphate, fluorouridine, 5-fluoro-2′-deoxycytidine, luoropyrimidine, 2′-deoxyfluorouridine, fluorocytosine, trifluoro-methyl-2′-deoxyuridine, cyclocytidine, arabinosyl cytosine, 5-aza-2′-deoxycytidine, arabinosyl-5-azacytosine, azacytidine, 6-azacytidine, N-phosphonoacetyl-L-asparticacid, pyrazofurin, 6-azauridine, azaribine, thymidine, fazarabine, and 3-deazauridine, cytarabine, gemcitabine, troxacitabine, decitabine, pseudoisocytidine, zebularine, ancitabine, capecitabine, N4-octadecyl-cytarabine, elaidic acid-cytarabine, cladribine, acyclovir, clofarabine, nelarabine, forodesine, 8-chloroadenosine, sapacitabine, thiarabine, and derivatives thereof. In embodiments, the nucleoside analog is 5-fluorouracil.

The administration of the IAP does not hinder the treatment of the cancer in the subject in accordance with embodiments of the present disclosure. In some embodiments, administration of the IAP allows reducing a length, dose and/or frequency of administration of the nucleoside analog. For example, in some embodiments, the use of IAP allows using a sub-therapeutic dose of the nucleoside analog. In some embodiments, the method in accordance with the present disclosure increases a therapeutic window of the nucleoside analog.

In some embodiments, administration of an IAP along with a chemotherapeutic treatment, prevents, eliminates, or reduces side effects associated with the chemotherapeutic treatment. Accordingly, in some aspects, the present disclosure provides a method of treating or preventing at least one side effect of a chemotherapeutic treatment with a nucleoside analog in a subject. The method comprises administering to the subject an IAP, wherein the subject has undergone or is undergoing a treatment of a cancer using the chemotherapeutic treatment. The side effect of the chemotherapeutic treatment can include one or more of diarrhea, colitis, mucositis, weight loss, pain, nausea, vomiting, constipation, anemia, malnutrition, alopecia, myelosuppression, renal toxicity, hair loss, numbness, changes in tastes, loss of appetite, thinned or brittle hair, mouth sores, memory loss, hemorrhage, cardiotoxicity, hepatotoxicity, ototoxicity, and post-chemotherapy cognitive impairment. The side effect of the chemotherapeutic treatment can comprise a gastrointestinal (GI) side effect, which can be caused by some chemotherapeutic treatments, such as, for example, nucleoside analogs (e.g., 5-fluorouracil). The method in accordance with the present disclosure allows treating or preventing GI side effects including mucositis and diarrhea.

### BRIEF DESCRIPTION OF THE DRAWINGS

FIG. 1A and 1B illustrate that administration of IAP reduces side effects of 5-FU. FIG. 1A shows assessment of weight change, as mean % of weight change ±SEM for untreated (top curve), vehicle+5-FU (middle curve), and SYN BIAPII+5-FU (bottom curve) mice versus a number of days. FIG. 1B shows assessment of stool consistency, as a mean stool consistency score ±SEM versus a number of days, of vehicle+5-FU and SYN BIAPII+5-FU mice relative to untreated mice data for which is shown as a baseline at the x-axis. As shown, the administration of 5-FU caused loose stools/diarrhea, and associated weight loss, in all of the treated animals, which peaked at day 6 and recovered by day 9. The administration of IAP shows a significantly faster recovery from the loose stools/diarrhea symptoms (p<0.03).

FIG. 2A, 2B, and 2C further illustrate that administration of IAP reduces side effects of 5-FU. FIG. 2A depicts the mean daily body weight through 50% group survival is displayed as percent change over time compared to body weight at Day 0+SEM. FIG. 2B shows the results of Stool Consistency Scoring daily over Days 0-12; mean scores+SEM are shown. FIG. 2C depicts a graphical representation of the percentage of total animal-days with diarrhea by treatment group.

### DETAILED DESCRIPTION

The present invention is based, inter alia, on the discovery that alkaline phosphatase (AP)-based agents, including without limitation intestinal alkaline phosphatase (IAP), can practically be used in conjunction with anti-cancer treatments that include the use of various chemotherapuetic treatments such as, for example, nucleoside analogs. The inventors have discovered that the use of IAP, which can be administered sequentially, simultaneously, or intermittedly with one or more chemotherapuetic treatments, or in a combination with one of these regimens, does not interfere with the action of the chemotherapeutic agent(s) and, more so, enhances the overall response of a subject to the chemotherapy, based on the discovery that IAP mitigates side effects of cancer treatments such as chemotherapy.

Chemotherapeutic exposure may lead to, for instance, gastrointestinal (GI) tract disruption, including, without limitation, dysbiosis, diarrhea, mucositis, leaky gut, endotoxemia, altered intestinal transit, malabsorption, and dysmotility. The present AP-based agents, including without limitation IAP, optionally orally administered, may reduce or prevent these and other side effects. Accordingly, the present invention relates, in various embodiments, to the treatment or prevention of the effects of chemotherapy with the present AP-based agents, including without limitation IAP, which can be administered orally. The inventors have discovered that IAP finds use in preventing, reducing, or eliminating chemotherapy-mediated GI side effects, such as diarrhea and/or mucositis colitis, in patients. Such use of IAP, as mentioned above, allows for more efficacious cancer treatment, as chemotherapy is not limited by dose- and regimen-limiting GI and other side effects.

Chemotherapuetic treatments involve use of various agents, including, without limitation, nucleoside analogs that are a class of antimetabolites widely used in the treatment of various malignancies, including blood cancers and solid tumors. The nucleoside analogs mimic physiological nucleosides and are incorporated into newly synthesized DNA, resulting in synthesis inhibition and chain termination. Some of the nucleoside analogs inhibit key enzymes involved in the generation of the purine and pyrimidine nucleotides and RNA synthesis, and directly activate the caspase cascade. The effects of the action of nucleoside analogs may lead to cell death. Non-limiting examples of the nucleoside analog include 5-fluorouracil, 5′-deoxyfluorouridine, 5-fluoro-2′-deoxyuridine triphosphate, fluorouridine, 5-fluoro-2′-deoxycytidine, luoropyrimidine, 2′-deoxyfluorouridine, fluorocytosine, trifluoro-methyl-2′-deoxyuridine, cyclocytidine, arabinosyl cytosine, 5-aza-2′-deoxycytidine, arabinosyl-5-azacytosine, azacytidine, 6-azacytidine, N-phosphonoacetyl-L-asparticacid, pyrazofurin, 6-azauridine, azaribine, thymidine, fazarabine, and 3-deazauridine, cytarabine, gemcitabine, troxacitabine, decitabine, pseudoisocytidine, zebularine, ancitabine, capecitabine, N4-octadecyl-cytarabine, elaidic acid-cytarabine, cladribine, acyclovir, clofarabine, nelarabine, forodesine, 8-chloroadenosine, sapacitabine, thiarabine, and derivatives thereof.

Most nucleoside analogs may cause potentially harmful side effects that include, but are not limited to, diarrhea and/mucositis. Other examples of side effects of a chemotherapeutic treatment with one or more nucleoside analogs comprise diarrhea, colitis, mucositis, weight loss, pain, nausea, vomiting, constipation, anemia, malnutrition, alopecia, myelosuppression, renal toxicity, hair loss, numbness, changes in tastes, loss of appetite, thinned or brittle hair, mouth sores, memory loss, hemorrhage, cardiotoxicity, hepatotoxicity, ototoxicity, and post-chemotherapy cognitive impairment.

IAP is an endogenous protein expressed by the intestinal epithelium that promotes GI homeostasis by detoxifying inflammatory mediators, and can be used to mitigate inflammation, tighten the gut barrier, and maintain gut homeostasis by promoting a healthy microbiome. For example, loss of IAP expression or function is associated with increased intestinal inflammation, dysbiosis, bacterial translocation, and systemic inflammation. Its primary functions, among others, in maintaining intestinal homeostasis are generally recognized as the regulation of bicarbonate secretion and duodenal surface pH, long chain fatty acid absorption, mitigation of intestinal inflammation through detoxification of pathogen-associated molecular patterns, and regulation of the gut microbiome. Several substrates that are acted on by IAP's phosphatase functions include, without wishing to be bound by theory, lipopolysaccharide (LPS), flagellin, CpG DNA, and nucleotide di-and tri-phosphates. Specifically, IAP is a target for therapeutics due to its ability to downregulate inflammation, regulate the microbiome, tighten the gut barrier through enhanced expression of claudins and occludins, and affect metabolism of adenosine tri-phosphate and diphosphate (ATP and ADP). The present invention contemplates a composition comprising IAP that does not hinder cancer treatment to the patient. In fact, according to the present invention, the methods described herein enhance the overall response of a subject to a chemotherapeutic treatment with a nucleoside analog.

Accordingly, the present invention is directed, in part, to a method for treating or preventing at least one side effect of a chemotherapeutic treatment with a nucleoside analog in a subject in need thereof, comprising administering to the subject an IAP, wherein the subject has undergone or is undergoing a treatment of a cancer using the chemotherapeutic treatment.

### Alkaline Phosphatases (APs)

The present disclosure is directed, in part, to pharmaceutical compositions, formulations, and uses of one or more alkaline phosphatase-based agents (AP-based agents). Alkaline phosphatases are dimeric metalloenzymes that catalyze the hydrolysis of phosphate esters and dephosphorylate a variety of target substrates at physiological and higher pHs. Illustrative APs that may be utilized in the present invention include, but are not limited to, IAP (e.g., calf IAP or bovine IAP, chicken IAP, goat IAP), placental alkaline phosphatase (PLAP), placental-like alkaline phosphatase, germ cell alkaline phosphatase (GCAP), tissue non-specific alkaline phosphatase (TNAP; which is primarily found in the liver, kidney, and bone), bone alkaline phosphatase, liver alkaline phosphatase, kidney alkaline phosphatase, bacterial alkaline phosphatase, fungal alkaline phosphatase, shrimp alkaline phosphatase, modified IAP, recombinant IAP, or any polypeptide comprising alkaline phosphatase activity.

In various embodiments, the present invention contemplates the use of mammalian alkaline phosphatases including, but are not limited to, IAP, placental alkaline phosphatase (PLAP), germ cell alkaline phosphatase (GCAP), and the tissue non-specific alkaline phosphatase (TNAP).

In some embodiments, the AP-based agent is IAP. IAP is produced in the proximal small intestine and it bound to the enterocytes via a GPI anchor. Some IAP is released into the intestinal lumen in conjunction with vesicles shed by the cells and as soluble protein stripped from the cells via phospholipases. The enzyme then traverses the small and large intestine such that some active enzyme can be detected in the feces. In an embodiment, the IAP is human IAP (hIAP).

In an embodiment, the IAP is calf IAP (cIAP), also known as bovine IAP (bIAP). There are multiple isozymes of bIAP, for example, with bIAP II and IV having higher specific activity than bIAP I. In an embodiment, the IAP is any one of the cIAP or bIAP isozymes (e.g., bIAP I, II, and IV). In an embodiment, the IAP is bIAP II. In another embodiment, the IAP is bIAP IV.

Also included within the definition of IAPs are IAP variants. An IAP variant has at least one or more amino acid modifications, generally amino acid substitutions, as compared to the parental wild-type sequence. In some embodiments, an IAP of the invention comprises an amino sequence having at least about 60% (e.g. about 60%, or about 61%, or about 62%, or about 63%, or about 64%, or about 65%, or about 66%, or about 67%, or about 68%, or about 69%, or about 70%, or about 71%, or about 72%, or about 73%, or about 74%, or about 75%, or about 76%, or about 77%, or about 78%, or about 79%, or about 80%, or about 81%, or about 82%, or about 83%, or about 84%, or about 85%, or about 86%, or about 87%, or about 88%, or about 89%, or about 90%, or about 91%, or about 92%, or about 93%, or about 94%, or about 95%, or about 96%, or about 97%, or about 98%, or about 99%) sequence identity with any of the sequences disclosed herein. In addition, IAP variants retain most or all of their biochemical activity, measured as described herein.

In various embodiments, the AP-based agent is hIAP or a variant thereof. In some embodiments, the AP-based agent is hIAP comprising the amino acid sequence of SEQ ID NO: 1 as depicted below.

HIAP
SEQ ID NO: 1

| | |
|---|---|
| 1 | mqgpwvllll glrlqlslgv ipaeeenpaf wnrqaaeald |
| | aakklqpiqk vaknlilflg |
| 61 | dglgyptvta trilkgqkng klgpetplam drfpylalsk |
| | tynydrqvpd saatataylc |
| 121 | gvkanfqtig lsaaarfnqc nttrgnevis vmnrakqagk |
| | svgvvtttrv qhaspagtya |
| 181 | htvnrnwysd admpasarqe gcqdiatqli snmdidvilg |
| | ggrkymfpmg tpdpeypada |
| 241 | sqngirldgk nlyqewlakh qgawyvwnrt elmqasldqs |
| | vthlmglfep gdtkyeihrd |
| 301 | ptldpslmem teaalrllsr nprgfylfve ggridhghhe |
| | gvayqaltea vmfddaiera |
| 361 | gqltseedtl tlytadhshv fsfggytlrg ssifglapsk |
| | aqdskaytsi lygngpgyvf |
| 421 | nsgyrpdyne sesgspdyqq qaavplsset hggedvavfa |
| | rgpqahlvhg vqeqsfvahv |
| 481 | mafaaclepy tacdlappac ttdaahpvaa slpllagtll |
| | llgasaap |

Without wishing to be bound by theory, it is believed that a cysteine at the carboxy terminus of the AP-based agent (e.g., at position 500 of SEQ ID NO: 1) may interfere with protein folding. Accordingly, in some embodiments, the AP-based agent includes a mutation of the cysteine (e.g., at position 500 of SEQ ID NO: 1). In some embodiments, the cysteine is replaced with glycine.

In various embodiments, the AP-based agent is bIAP II or a variant thereof. In an embodiment, the bIAP II comprises the signal peptide and carboxy terminus of bIAP I. In an embodiment, the bIAP II comprises an aspartate and position 248 (similar to bIAP IV). In an embodiment, the bIAP II comprises the amino acid sequence of SEQ ID NO: 2:

BIAP II with 248D assignment.
The signal peptide and sequence past 480 are
derived from bIAP I
SEQ ID NO: 2

| | |
|---|---|
| 1 | mqgacvllll glhlqlslgl ipaeeenpaf wnrgaagald |
| | vakklqpiqt aaknvilflg |
| 61 | dgmgvptvta trilkgqmng klgpetplam dqfpyvalsk |
| | tynvdrqvpd sagtataylc |
| 121 | gvkgnyrtig vsaaarynqc nttrgnevts vinrakkagk |
| | avgvvtttry qhaspagaya |
| 181 | htvnrnwysd adlpadaqkn gcgdiaaglv ynmdidvilg |
| | ggrmymfpeg tpdpeypdda |
| 241 | svngvrkdkq nlvqewqakh qgagyvwnrt allqaaddss |
| | vthlmglfep admkynvqqd |
| 301 | htkdptlaem teaalqvlsr nprgfylfve ggridhghhd |
| | gkaymaltea imfdnaiaka |
| 361 | neltseldtl ilvtadhshv fsfggytlrg tsifglapgk |
| | aldsksytsi lygngpgyal |
| 421 | gggsrpdvng stseepsyrq qaavplaset hggedvavfa |
| | rgpqahlvhg vqeetfvahi |
| 481 | mafagcvepy tdcnlpapat atsipdaahl aasppplall |
| | agamllllap tly |

In various embodiments, the bIAP II comprises the amino acid sequence of SEQ ID NO: 41.

BIAP II with stop codon and no leader sequence
(SYN-020) (SEQ ID NO: 41):
LIPAEEENPAFWNRQAAQALDVAKKLQPIQTAAKNVILFLGDGMGVPTVT
ATRILKGQMNGKLGPETPLAMDQFPYVALSKTYNVDRQVPDSAGTATAYL
CGVKGNYRTIGVSAAARYNQCNTTRGNEVTSVINRAKKAGKAVGVVTTTR
VQHASPAGAYAHTVNRNWYSDADLPADAQKNGCQDIAAQLVYNMDIDVIL
GGGRMYMFPEGTPDPEYPDDASVNGVRKDKQNLVQEWQAKHQGAQYVWNR
TALLQAADDSSVTHLMGLFEPADMKYNVQQDHTKDPTLAEMTEAALQVLS
RNPRGFYLFVEGGRIDHGHHDGKAYMALTEAIMFDNAIAKANELTSELDT
LILVTADHSHVFSFGGYTLRGTSIFGLAPGKALDSKSYTSILYGNGPGYA
LGGGSRPDVNGSTSEEPSYRQQAAVPLASETHGGEDVAVFARGPQAHLVH
GVQEETFVAHIMAFAGCVEPYTDCNLPAPATATSIPD

In various embodiments, the AP-based agent is bIAP IV or a variant thereof. In an embodiment, the bIAP IV comprises the amino acid sequence of SEQ ID NO: 3:

BIAP IV
SEQ ID NO: 3

| | |
|---|---|
| 1 | mgwacvllll glwlqlsltf ipaeeedpaf wnrgaagald |
| | vakklqpiqt aaknvilflg |
| 61 | dgmgvptvta trilkgqmng klgpetplam dqfpyvalsk |
| | tynvdrqvpd sagtataylc |
| 121 | gvkgnyktig vsaaarynqc nttsgnevts vmnrakkagk |
| | svgvvttsrv qhaspagaya |
| 181 | htvnrnwysd adlpadaqty gcqdiatqlv nnmdidvilg |
| | ggrmymfpeg tpdpeypydv |
| 241 | nqtgvrkdkr nlvqewqakh qgagyvwnrt ellqaandps |
| | vthlmglfep admkynvqqd |
| 301 | ptkdptleem teaalqvlsr npqgfylfve ggridhghhe |
| | gkaymaltdt vmfdnaiaka |
| 361 | neltseldtl ilatadhshv fsfggytlrg tsifglapsk |
| | asdnksytsi lygngpgyvl |
| 421 | ggglrpdvnd sisedpsyrq qaavplsses hggedvavfa |
| | rgpqahlvhg vqeetfvahv |
| 481 | mafagcvepy tdcnlpapsg lsdaahlaas ppslallaga |
| | mllllapaly |

Mammalian alkaline phosphatases are glycosylphosphatidyl-inositol (GPI) anchored proteins. They have signal peptides and are translated into the secretory pathway. Once in the endoplasmic reticulum (ER), the proteins are glycosylated and folded. There are two disulfide bonds as well as a single free cysteine that is apparently not accessible on the surface. In the late ER, the carboxy terminus is removed and the GPI anchor is appended. GPI anchoring is therefore a process that occurs at the carboxy terminus of the alkaline phosphatase. The inclusion of stop codons at the anchor site enables secretion of biologically active protein (presumably the homodimer). While there is no consensus sequence, the carboxy terminus includes three amino acids, termed omega, omega +1, and omega +2 which are followed by a short stretch of hydrophilic amino acids and then a stretch of hydrophobic amino acids. Without wishing to be bound by theory, it is believed that the hydrophobicity is critical for embedding the carboxy terminus in the ER membrane. Then an enzymatic reaction replaces the carboxy terminus with the GPI anchor.

Within hPLAP, the GPI anchor is attached at an aspartate in the sequence, DAAH. Similarly, hIAP, bIAP II, and bIAP IV also have this DAAH sequence conserved, potentially serving as the GPI anchor site. Mutational studies with hPLA indicate that preventing GPI anchoring results in intracellular retention. In addition, mutations around the anchor site or in the hydrophobic domain either 1) prevent anchor attachment leading to intracellular retention or 2) do not block anchor attachment. Without wishing to be bound by theory, it is believed that the hydrophobic domain serves as a signal for GPI anchor attachment. Truncating or eliminating the hydrophobic domain leads to secretion. Finally, there is a single mutation in the hydrophobic domain that, in hPLAP, enables secretion of a protein with its hydrophobic domain intact.

In various embodiments, the AP-based agent of the invention is GPI anchored to a host cell. For example, the AP-based agent may be GPI anchored to the cell membrane of the host cell. In other embodiments, the AP-based agent of the invention is a secreted rather than an anchored protein. In some embodiments, the AP-based agent is not GPI anchored. In some embodiments, the AP-based agent may lack the GPI anchor site. In some embodiments, the AP-based agent comprises a stop codon that is inserted immediately after the GPI anchor site. In an embodiment, the AP-based agent comprises a stop codon after the aspartate in the DAAH consensus site (e.g., at amino acid 503 of hIAP and bIAP IV or amino acid 506 of bIAP II).

HIAP with stop codon
(SEQ ID NO: 4)

| | |
|---|---|
| 1 | mqgpwvllll glrlqlslgv ipaeeenpaf wnrqaaeald |
| | aakklqpiqk vaknlilflg |
| 61 | dglgvptvta trilkgqkng klgpetplam drfpylalsk |
| | tynvdrqvpd saatataylc |
| 121 | gvkanfqtig lsaaarfnqc nttrgnevis vmnrakqagk |
| | svgvvtttrv qhaspagtya |
| 181 | htvnrnwysd admpasarqe gcqdiatqli snmdidvilg |
| | ggrkymfpmg tpdpeypada |
| 241 | sqngirldgk nlvqewlakh qgawyvwnrt elmqasldqs |
| | vthlmglfep gdtkyeihrd |
| 301 | ptldpslmem teaalrllsr nprgfylfve ggridhghhe |
| | gvayqaltea vmfddaiera |
| 361 | gqltseedtl tlvtadhshv fsfggytlrg ssifglapsk |
| | aqdskaytsi lygngpgyvf |
| 421 | nsgvrpdvne sesgspdyqq qaavplsset hggedvavfa |
| | rgpqahlvhg vqeqsfvahv |
| 481 | mafaaclepy tacdlappag ttd |

BIAP II with stop codon
(SEQ ID NO: 5)

| | |
|---|---|
| 1 | mqgacvllll glhlqlslgl ipaeeenpaf wnrqaaqald |
| | vakklqpiqt aaknvilflg |
| 61 | dgmgvptvta trilkgqmng klgpetplam dqfpyvalsk |
| | tynvdrqvpd sagtataylc |
| 121 | gvkgnyrtig vsaaarynqc nttrgnevts vinrakkagk |
| | avgvvtttrv qhaspagaya |
| 181 | htvnrnwysd adlpadaqkn gcqdiaaqlv ynmdidvilg |
| | ggrmymfpeg tpdpeypdda |
| 241 | svngvrkdkq nlvqewqakh qgaqyvwnrt allqaaddss |
| | vthlmglfep admkynvqqd |
| 301 | htkdptlaem teaalqvlsr nprgfylfve ggridhghhd |
| | gkaymaltea imfdnaiaka |
| 361 | neltseldtl ilvtadhshv fsfggytlrg tsifglapgk |
| | aldsksytsi lygngpgyal |
| 421 | gggsrpdvng stseepsyrq qaavplaset hggedvavfa |
| | rgpqahlvhg vqeetfvahi |
| 481 | mafagcvepy tdcnlpapat atsipd |

BIAP IV with stop codon
(SEQ ID NO: 6)

| | |
|---|---|
| 1 | mqwacvllll glwlqlsltf ipaeeedpaf wnrqaaqald |
| | vakklqpiqt aaknvilflg |
| 61 | dgmgvptvta trilkgqmng klgpetplam dqfpyvalsk |
| | tynvdrqvpd sagtataylc |
| 121 | gvkgnyktig vsaaarynqc nttsgnevts vmnrakkagk |
| | svgvvttsry qhaspagaya |
| 181 | htvnrnwysd adlpadaqty gcqdiatqlv nnmdidvilg |
| | ggrmymfpeg tpdpeypydv |
| 241 | nqtgvrkdkr nlvqewqakh qgaqyvwnrt ellqaandps |
| | vthlmglfep admkynvqqd |
| 301 | ptkdptleem teaalqvlsr npqgfylfve ggridhghhe |
| | gkaymaltdt vmfdnaiaka |
| 361 | neltseldtl ilatadhshv fsfggytlrg tsifglapsk |
| | asdnksytsi lygngpgyvl |
| 421 | ggglrpdvnd sisedpsyrq qaavplsses hggedvavfa |
| | rgpqahlvhg vqeetfvahv |
| 481 | mafagcvepy tdcnlpapsg lsd |

In an embodiment, the AP-based agent is bIAP IV and includes a stop codon after amino acid 508 to mimic a secreted PLAP construct as depicted below:

BIAP IV with stop codon after amino acid 508
(SEQ ID NO: 7)

| | |
|---|---|
| 1 | mgwacvllll glwlqlsltf ipaeeedpaf wnrgaagald |
| | vakklqpiqt aaknvilflg |
| 61 | dgmgvptvta trilkgqmng klgpetplam dqfpyvalsk |
| | tynvdrqvpd sagtataylc |
| 121 | gvkgnyktig vsaaarynqc nttsgnevts vmnrakkagk |
| | svgvvttsry qhaspagaya |
| 181 | htvnrnwysd adlpadaqty gcqdiatqlv nnmdidvilg |
| | ggrmymfpeg tpdpeypydv |
| 241 | nqtgvrkdkr nlvqewqakh qgagyvwnrt ellqaandps |
| | vthlmglfep admkynvqqd |
| 301 | ptkdptleem teaalqvlsr npqgfylfve ggridhghhe |
| | gkaymaltdt vmfdnaiaka |
| 361 | neltseldtl ilatadhshv fsfggytlrg tsifglapsk |
| | asdnksytsi lygngpgyvl |
| 421 | ggglrpdynd sisedpsyrq qaavplsses hggedvavfa |
| | rgpqahlvhg vqeetfyahv |
| 481 | mafagcvepy tdcnlpapsg lsdaahla |

In various embodiments, the AP-based agent of the invention is a fusion protein. In some embodiments, the AP-based agent comprises an alkaline phosphatase fused to a protein domain that replaces the GPI anchor sequence. In some embodiments, the alkaline phosphatase is fused to a protein domain that promotes protein folding and/or protein purification and/or protein dimerization and/or protein stability. In various embodiments, the AP-based agent fusion protein has an extended serum half-life.

In an embodiment, the alkaline phosphatase is fused to an immunoglobulin Fc domain and/or hinge region. In various embodiments, the immunoglobulin Fc domain and/or hinge region is derived from the Fc domain and/or hinge region of an antibody (e.g., of IgG, IgA, IgD, and IgE, inclusive of subclasses (e.g. IgG1, IgG2, IgG3, and IgG4, and IgA1 and IgA2)). In an embodiment, the AP-based agent of the invention comprises an alkaline phosphatase fused to the hinge region and/or Fc domain of IgG.

In various embodiments, the AP-based agent is fused to a Fc domain of IgG comprising one or more mutations. In some embodiments, the one or more mutations in the Fc domain of IgG function to increase serum half-life and longevity. In some embodiments, the Fc domain of IgG comprises one or more mutations at amino acid residues 251-256, 285-290, 308-314, 385-389 and 428-436, numbered according to the EU index as in Kabat (see Kabat et al., (1991) Sequences of Proteins of Immunological Interest, U.S. Public Health Service, National Institutes of Health, Washington, DC). In some embodiments, at least one of the amino acid substitutions is at amino acid residue 252, 254, 256, 309, 311, 433 or 434. In an embodiment, the amino acid substitution at amino acid residue 252 is a substitution with tyrosine, phenylalanine, tryptophan or threonine. In an embodiment, the amino acid substitution at amino acid residue 254 is a substitution with threonine. In an embodiment, the amino acid substitution at amino acid residue 256 is a substitution with serine, arginine, glutamine, glutamic acid, aspartic acid, or threonine. In an embodiment, the amino acid substitution at amino acid residue 309 is a substitution with proline. In an embodiment, the amino acid substitution at amino acid residue 311 is a substitution with serine. In an embodiment, the amino acid substitution at amino acid residue 385 is a substitution with arginine, aspartic acid, serine, threonine, histidine, lysine, alanine or glycine. In an embodiment, the amino acid substitution at amino acid residue 386 is a substitution with threonine, proline, aspartic acid, serine, lysine, arginine, isoleucine, or methionine. In an embodiment, the amino acid substitution at amino acid residue 387 is a substitution with arginine, proline, histidine, serine, threonine, or alanine. In an embodiment, the amino acid substitution at amino acid residue 389 is a substitution with proline, serine or asparagine. In an embodiment, the amino acid substitution at amino acid residue 433 is a substitution with arginine, serine, isoleucine, proline, or glutamine. In an embodiment, the amino acid substitution at amino acid residue 434 is a substitution with histidine, phenylalanine, or tyrosine.

In some embodiments, the Fc domain of IgG comprises one or more mutations at amino acid residue 252, 254, 256, 433, 434, or 436. In an embodiment, the Fc domain of IgG includes a triple M252Y/S254T/T256E mutation or YTE mutation. In another embodiment, the Fc domain of IgG includes a triple H433K/N434F/Y436H mutation or KFH mutation. In a further embodiment, the Fc domain of IgG includes a YTE and KFH mutation in combination.

In some embodiments, the Fc domain of IgG contains one or more mutations at amino acid residues 250, 253, 307, 310, 380, 428, 433, 434, and 435. Exemplary mutations include T250Q, M428L, T307A, E380A, 1253A, H310A, M428L, H433K, N434A, N434F, N434S, and H435A. In an embodiment, the Fc domain of IgG comprises a M428L/N434S mutation or LS mutation. In another embodiment, the Fc domain of IgG comprises a T250Q/M428L mutation or QL mutation. In another embodiment, the Fc domain of IgG comprises an N434A mutation. In another embodiment, the Fc domain of IgG comprises a T307A/E380A/N434A mutation or AAA mutation. In another embodiment, the Fc domain of IgG comprises an 1253A/H310A/H435A mutation or IHH mutation. In another embodiment, the Fc domain of IgG comprises a H433K/N434F mutation. In another embodiment, the Fc domain of IgG region comprises a M252Y/S254T/T256E and a H433K/N434F mutation in combination.

Exemplary mutations in the Fc domain of IgG are described, for example, in Robbie, et al., Antimicrobial Agents and Chemotherapy (2013), 57(12):6147-6153, Dall'Acqua et al., JBC (2006), 281(33):23514-24, Dall'Acqua et al., Journal of Immunology (2002), 169:5171-80, Ko et al. Nature (2014) 514:642-645, Grevys et al Journal of Immunology. (2015), 194(11):5497-508, and U.S. Pat. No. 7,083,784, the entire contents of which are hereby incorporated by reference.

In various embodiments, the one or more mutations in the Fc domain of IgG increases affinity for the neonatal Fc receptor (FcRn). In some embodiments, the one or more mutations in the Fc domain of IgG increases affinity for FcRn at a pH of about 6.0, about 6.1, about 6.2, about 6.3, about 6.4, or about 6.5.

In various embodiments, the alkaline phosphatase is fused to one or more of PEG, XTENylation (e.g., as rPEG), polysialic acid (POLYXEN), albumin, elastin-like protein, elastin like protein (ELP), PAS, HAP, GLK, CTP, and transferrin. In various embodiments, the alkaline phosphatase is fused to one or more of the agents described in BioDrugs (2015) 29:215-239, the entire contents of which are hereby incorporated by reference.

In an embodiment, the alkaline phosphatase is fused to a protein domain (e.g., an immunoglobulin Fc domain) via a linker to the GPI anchor site. For example, the alkaline phosphatase may be fused to a protein domain via the aspartate at the GPI anchor sequence. The invention contemplates the use of a variety of linker sequences. In various embodiments, the linker may be derived from naturally-occurring multi-domain proteins or are empirical linkers as described, for example, in Chichili et al., (2013), Protein Sci. 22(2): 153-167, Chen et al., (2013), Adv Drug Deliv Rev. 65(10): 1357-1369, the entire contents of which are hereby incorporated by reference. In some embodiments, the linker may be designed using linker designing databases and computer programs such as those described in Chen et al., (2013), Adv Drug Deliv Rev. 65(10): 1357-1369 and Crasto et al., (2000), Protein Eng. 13(5): 309-312, the entire contents of which are hereby incorporated by reference. In various embodiments, the linker may be functional. For example, without limitation, the linker may function to improve the folding and/or stability, improve the expression, improve the pharmacokinetics, and/or improve the bioactivity of the present AP-based agent. In another example, the linker may function to target the AP-based agent to a particular cell type or location.

In some embodiments, the linker is a polypeptide. In some embodiments, the linker is less than about 100 amino acids long. For example, the linker may be less than about 100, about 95, about 90, about 85, about 80, about 75, about 70, about 65, about 60, about 55, about 50, about 45, about 40, about 35, about 30, about 25, about 20, about 19, about 18, about 17, about 16, about 15, about 14, about 13, about 12, about 11, about 10, about 9, about 8, about 7, about 6, about 5, about 4, about 3, or about 2 amino acids long. In some embodiments, the linker is flexible. In another embodiment, the linker is rigid.

In various embodiments, the linker is substantially comprised of glycine and serine residues (e.g. about 30%, or about 40%, or about 50%, or about 60%, or about 70%, or about 80%, or about 90%, or about 95%, or about 97% glycines and serines). In an embodiment, the linker sequence is GGSGGSGGGGSGGGGS (SEQ ID NO: 19). Additional illustrative linkers include, but are not limited to, linkers having the sequence LE, GGGGS (SEQ ID NO: 20), (GGGGS)ₙ (n=2-4) (SEQ ID NO: 21-23). (Gly)₈ (SEQ ID NO: 24), (Gly)₆ (SEQ ID NO: 25), (EAAAK)ₙ (n=1-3) ((SEQ ID NO: 26-28), A(EAAAK)ₙA (n=2-5) (SEQ ID NO: 29-32), AEAAAKEAAAKA (SEQ ID NO: 30), A(EAAAK)₄ALEA(EAAAK)₄A (SEQ ID NO: 33), PAPAP (SEQ ID NO: 34), KESGSVSSEQLAQFRSLD (SEQ ID NO: 35), EGKSSGSGSESKST (SEQ ID NO: 36), GSAGSAAGSGEF (SEQ ID NO: 37), and (XP)ₙ, with X designating any amino acid, e.g., Ala, Lys, or Glu. In various embodiments, the linker is GGS.

In some embodiments, the linker is a hinge region of an antibody (e.g., of IgG, IgA, IgD, and IgE, inclusive of subclasses (e.g. IgG1, IgG2, IgG3, and IgG4, and IgA1 and IgA2)). In various embodiments, the linker is a hinge region of an antibody (e.g., of IgG, IgA, IgD, and IgE, inclusive of subclasses (e.g. IgG1, IgG2, IgG3, and IgG4, and IgA1 and IgA2). The hinge region, found in IgG, IgA, IgD, and IgE class antibodies, acts as a flexible spacer, allowing the Fab portion to move freely in space. In contrast to the constant regions, the hinge domains are structurally diverse, varying in both sequence and length among immunoglobulin classes and subclasses. For example, the length and flexibility of the hinge region varies among the IgG subclasses. The hinge region of IgG1 encompasses amino acids 216-231 and, because it is freely flexible, the Fab fragments can rotate about their axes of symmetry and move within a sphere centered at the first of two inter-heavy chain disulfide bridges. IgG2 has a shorter hinge than IgG1, with 12 amino acid residues and four disulfide bridges. The hinge region of IgG2 lacks a glycine residue, is relatively short, and contains a rigid poly-proline double helix, stabilized by extra inter-heavy chain disulfide bridges. These properties restrict the flexibility of the IgG2 molecule. IgG3 differs from the other subclasses by its unique extended hinge region (about four times as long as the IgG1 hinge), containing 62 amino acids (including 21 prolines and 11 cysteines), forming an inflexible poly-proline double helix. In IgG3, the Fab fragments are relatively far away from the Fc fragment, giving the molecule a greater flexibility. The elongated hinge in IgG3 is also responsible for its higher molecular weight compared to the other subclasses. The hinge region of IgG4 is shorter than that of IgG1 and its flexibility is intermediate between that of IgG1 and IgG2. The flexibility of the hinge regions reportedly decreases in the order IgG3>IgG1>IgG4>IgG2.

According to crystallographic studies, the immunoglobulin hinge region can be further subdivided functionally into three regions: the upper hinge region, the core region, and the lower hinge region. See Shin et al., 1992 Immunological Reviews 130:87. The upper hinge region includes amino acids from the carboxyl end of CHI to the first residue in the hinge that restricts motion, generally the first cysteine residue that forms an interchain disulfide bond between the two heavy chains. The length of the upper hinge region correlates with the segmental flexibility of the antibody. The core hinge region contains the inter-heavy chain disulfide bridges, and the lower hinge region joins the amino terminal end of the C_{H2} domain and includes residues in C_{H2}. The core hinge region of wild-type human IgG1 contains the sequence Cys-Pro-Pro-Cys which, when dimerized by disulfide bond formation, results in a cyclic octapeptide believed to act as a pivot, thus conferring flexibility. In various embodiments, the present linker comprises, one, or two, or three of the upper hinge region, the core region, and the lower hinge region of any antibody (e.g., of IgG, IgA, IgD, and IgE, inclusive of subclasses (e.g. IgG1, IgG2, IgG3, and IgG4, and IgA1 and IgA2). The hinge region may also contain one or more glycosylation sites, which include a number of structurally distinct types of sites for carbohydrate attachment. For example, IgA1 contains five glycosylation sites within a 17-amino-acid segment of the hinge region, conferring resistance of the hinge region polypeptide to intestinal proteases, considered an advantageous property for a secretory immunoglobulin. In various embodiments, the linker of the present invention comprises one or more glycosylation sites.

In some embodiments, the linker is a synthetic linker such as PEG.

Illustrative Fc fusion constructs of the invention include:

| BIAP II with Fc Fusion - Fc domain is underlined | |
|---|---|
| (SEQ ID NO: 8) | |
| 1 | mqgacvllll glhlqlslgl ipaeeenpaf wnrqaaqald vakklqpiqt aaknvilflg |
| 61 | dgmgvptvta trilkgqmng klgpetplam dqfpyvalsk tynvdrqvpd sagtataylc |
| 121 | gvkgnyrtig vsaaarynqc nttrgnevts vinrakkagk avgvvtttry qhaspagaya |
| 181 | htvnrnwysd adlpadaqkn gcgdiaaglv ynmdidvilg ggrmymfpeg tpdpeypdda |
| 241 | svngvrkdkq nlvqewqakh qgagyvwnrt allqaaddss vthlmglfep admkynvqqd |
| 301 | htkdptlaem teaalqvlsr nprgfylfve ggridhghhd gkaymaltea imfdnaiaka |
| 361 | neltseldtl ilvtadhshv fsfggytlrg tsifglapgk aldsksytsi lygngpgyal |
| 421 | gggsrpdvng stseepsyrq qaavplaset hggedvavfa rgpqahlvhg vqeetfvahi |
| 481 | mafagcvepy tdcnlpapat atsipdGGSGGSGGGGSGGGGSEPKSCDKTHTCPPCPAPE |
| | LLGGPSVFLFPPKPKDTLMI SRTPEVTCVVVDVSHEDPQV KFNWYVDGVQVHNAKTKPRE |
| | QQYNSTYRVVSVLTVLHQNW LDGKEYKCKVSNKALPAPIE KTISKAKGQPREPQVYTLPP |
| | SREEMTKNQVSLTCLVKGFY PSDIAVEWESNGQPENNYKT TPPVLDSDGSFFLYSKLTVD |
| | KSRWQQGNVFSCSVMHEALH NHYTQKSLSLSPGK |
| BIAP IV with Fc Fusion - Fc domain is underlined | |
| (SEQ ID NO: 9) | |
| 1 | mgwacvllll glwlqlsltf ipaeeedpaf wnrgaagald vakklqpiqt aaknvilflg |
| 61 | dgmgvptvta trilkgqmng klgpetplam dqfpyvalsk tynvdrqvpd sagtataylc |
| 121 | gvkgnyktig vsaaarynqc nttsgnevts vmnrakkagk svgvvttsry qhaspagaya |
| 181 | htvnrnwysd adlpadaqty gcqdiatqlv nnmdidvilg ggrmymfpeg tpdpeypydv |
| 241 | nqtgvrkdkr nlvqewqakh qgagyvwnrt ellqaandps vthlmglfep admkynvqqd |
| 301 | ptkdptleem teaalqvlsr npqgfylfve ggridhghhe gkaymaltdt vmfdnaiaka |
| 361 | neltseldtl ilatadhshv fsfggytlrg tsifglapsk asdnksytsi lygngpgyvl |
| 421 | ggglrpdynd sisedpsyrq qaavplsses hggedvavfa rgpqahlvhg vqeetfyahv |
| 481 | mafagcvepy tdcnlpapsg lsdGGSGGSGGGGSGGGGSEPKSCDKTHTCPPCPAPE |
| | LLGGPSVFLFPPKPKDTLMI SRTPEVTCVVVDVSHEDPQV KFNWYVDGVQVHNAKTKPRE |
| | QQYNSTYRVVSVLTVLHQNW LDGKEYKCKVSNKALPAPIE KTISKAKGQPREPQVYTLPP |
| | SREEMTKNQVSLTCLVKGFY PSDIAVEWESNGQPENNYKT TPPVLDSDGSFFLYSKLTVD |
| | KSRWQQGNVFSCSVMHEALH NHYTQKSLSLSPGK |
| HIAP with Fc Fusion - Fc domain is underlined: | |
| SEQ ID NO: 18 | |
| 1 | mqgpwvllll glrlqlslgy ipaeeenpaf wnrqaaeald aakklqpiqk vaknlilflg |
| 61 | dglgyptvta trilkgqkng klgpetplam drfpylalsk tynydrqvpd saatataylc |
| 121 | gvkanfqtig lsaaarfnqc nttrgnevis ymnrakgagk svgyytttry qhaspagtya |
| 181 | htvnrnwysd admpasarqe gcgdiatqli snmdidvilg ggrkymfpmg tpdpeypada |
| 241 | sqngirldgk nlygewlakh qgawyywnrt elmgasldqs vthlmglfep gdtkyeihrd |
| 301 | ptldpslmem teaalrllsr nprgfylfve ggridhghhe gvaygaltea vmfddaiera |
| 361 | gqltseedtl tlytadhshy fsfggytlrg ssifglapsk aqdskaytsi lygngpgyvf |
| 421 | nsgyrpdyne sesgspdygq qaavplsset hggedvavfa rgpqahlvhg vqeqsfvahv |
| 481 | mafaaclepy tacdlappac ttdaahpvaa slpllagtll llgasaap |
| | GGSGGSGGGGSGGGGSEPKSCDKTHTCPPCPAPE LLGGPSVFLFPPKPKDTLMI |
| | SRTPEVTCVVVDVSHEDPQV KFNWYVDGVQVHNAKTKPRE QQYNSTYRVVSVLTVLHQNW |
| | LDGKEYKCKVSNKALPAPIE KTISKAKGQPREPQVYTLPP SREEMTKNQVSLTCLVKGFY |
| | PSDIAVEWESNGQPENNYKT TPPVLDSDGSFFLYSKLTVD KSRWQQGNVFSCSVMHEALH |
| | NHYTQKSLSLSPGK |

In various embodiments, the linker can be substituted with any other linker disclosed herein.

A Saccharomyces alkaline phosphatase, Pho8, is produced as an inactive pro-enzyme. It is not GPI anchored, but is a transmembrane protein with its amino terminus extending out of a lysosome into the cytoplasm. Within the lysosome, an enzyme, PEP4, cleaves the carboxy terminus to activate the enzyme. Without wishing to be bound by theory, it is believed that mammalian alkaline phosphatases may also be generated as inactive pro-enzymes. This is because alkaline phosphatases can dephosphorylate ATP, so that activity in the ER could drain the ER of its major energy source. Without wishing to be bound by theory, it is believed that the inhibitory function is located to the carboxy terminus that would be relieved upon GPI anchor addition. Alternatively, other activities such as folding or metal (Zn or Mg) inclusion could control activity.

In various embodiments, the AP-based agent of the invention is a pro-enzyme. In an embodiment, the activity of the proenzyme is suppressed by a carboxy terminus. In an embodiment, protease removal of the carboxy terminus reactivates the enzymatic activity of the alkaline phosphatase. In an embodiment, the pro-enzyme is more efficiently secreted than the enzyme without the carboxy terminus.

In some embodiments, for generation of the pro-enzyme, the native carboxy terminus of the alkaline phosphatase is replaced with the analogous sequence from hPLAP. In some embodiments, a mutation is made in the hydrophobic carboxy tail to promote protein secretion without cleavage of the carboxy terminus. In an illustrative embodiment, a single point mutation such as a substitution of leucine with e.g., arginine is generated in the hydrophobic carboxy terminus (e.g. allpllagtl is changed to e.g., allplragtl) to result in secretion of the enzyme without removal of the carboxy terminus.

In an embodiment, the AP-based agent is altered to include a specific enzyme cleavage site which allows subsequent removal of the carboxy terminus. In an embodiment, the AP-based agent includes a protease cleavage site. Illustrative protease cleavage sites include, but are not limited to, cleavage sites recognized by furin, Rhinovirus 16 3C protease, factor Xa protease, trpysin, chymotrypsin, elastase, pepsin, papain subtilisin, thermolysin, V-8 protease, submaxillaris protease, clostripain, thrombin, collagenase, and any other endoproteases. In an alternative embodiment, the AP-based agent includes a cleavage site recognized by a digestive enzyme present in the GI tract. In such embodiments, the AP-based agent may be administered as a pro-drug that is subsequently activated in the GI tract.

In an illustrative embodiment, the proenzyme is a proenzyme of bIAP IV having the following sequences:

BIAP IV with the hPLAP Carboxy Terminus and
Mutation for Unprocessed Secretion and RV3C
Cleavage (at . . . LEVLFQGP . . . ):
SEQ ID NO: 10

| | |
|---|---|
| 1 | mqwacvllll glwlqlsltf ipaeeedpaf wnrqaaqald |
| | vakklqpiqt aaknvilflg |
| 61 | dgmgvptvta trilkgqmng klgpetplam dqfpyvalsk |
| | tynvdrqvpd sagtataylc |
| 121 | gvkgnyktig vsaaarynqc nttsgnevts vmnrakkagk |
| | svgvvttsrv qhaspagaya |
| 181 | htvnrnwysd adlpadaqty gcqdiatqlv nnmdidvilg |
| | ggrmymfpeg tpdpeypydv |
| 241 | nqtgvrkdkr nlvqewqakh qgagyvwnrt ellqaandps |
| | vthlmglfep admkynvqqd |
| 301 | ptkdptleem teaalqvlsr npqgfylfve ggridhghhe |
| | gkaymaltdt vmfdnaiaka |
| 361 | neltseldtl ilatadhshv fsfggytlrg tsifglapsk |
| | asdnksytsi lygngpgyvl |
| 421 | ggglrpdvnd sisedpsyrq qaavplsses hggedvavfa |
| | rgpqahlvhg vqeetfvahv |
| 481 | mafagcvepy tdcnlevlfq gpappagttd aahpgrsvvp |
| | allplragtl llletatap |

BIAP IV with hPLAP Carboxy Terminus and Mutation
for Unprocessed Secretion and FXa Cleavage
(at . . . IEGR . . . ):
SEQ ID NO: 11

| | |
|---|---|
| 1 | mqwacvllll glwlqlsltf ipaeeedpaf wnrqaaqald |
| | vakklqpiqt aaknvilflg |
| 61 | dgmgvptvta trilkgqmng klgpetplam dqfpyvalsk |
| | tynvdrqvpd sagtataylc |
| 121 | gvkgnyktig vsaaarynqc nttsgnevts vmnrakkagk |
| | svgvvttsry qhaspagaya |
| 181 | htvnrnwysd adlpadaqty gcqdiatqlv nnmdidvilg |
| | ggrmymfpeg tpdpeypydv |
| 241 | nqtgvrkdkr nlvqewqakh qgagyvwnrt ellqaandps |
| | vthlmglfep admkynvqqd |
| 301 | ptkdptleem teaalqvlsr npqgfylfve ggridhghhe |
| | gkaymaltdt vmfdnaiaka |
| 361 | neltseldtl ilatadhshv fsfggytlrg tsifglapsk |
| | asdnksytsi lygngpgyvl |
| 421 | ggglrpdvnd sisedpsyrq qaavplsses hggedvavfa |
| | rgpqahlvhg vqeetfvahv |
| 481 | mafagcvepy tdcnlappag ttdaahpieg rsvvpallpl |
| | ragtllllet atap |

In various embodiments, the AP-based agent of the invention is efficiently expressed and secreted from a host cell. In an embodiment, the AP-based agent of the invention is efficiently transcribed in the host cell. In another embodiment, the AP-based agent exhibits enhanced RNA stability and/or transport in the host cell. In another embodiment, the AP-based agent is efficiently translated in the host cell. In a further embodiment, the AP-based agent is efficiently folded and/or transits efficiently through the ER, pre-golgi, and golgi. In another embodiment, the AP-based agent exhibits enhanced protein stability.

In various embodiments, the AP-based agent of the invention is GPI anchored to the cell membrane of a host cell. In other embodiments, the AP-based agent is secreted from the host cell. In such embodiments, the AP-based agent may include a protease cleavage site just upstream from the GPI anchor site. Illustrative protease cleavage sites are described previously. In an embodiment, the protease cleavage site is a furin cleavage site. In another embodiment, the AP-based agent may include a cleavage site recognized by a digestive enzyme in the GI tract just upstream from the GPI anchor site. In these embodiments, the AP-based agent is anchored in the ER and released in the late golgi and secreted.

In various embodiments, the AP-based agents are efficiently expressed in a host cell. In an embodiment, the Kozak sequence of the DNA construct encoding the AP-based agent is optimized. The Kozak sequence is the nucleotide sequence flanking the ATG start codon that instructs the ribosome to start translation. There is flexibility in the design of a Kozak sequence, but one canonical sequence is GCCGCCACCATGG (SEQ ID NO: 38). The purine in the −3 position and the G in the +4 position are the most important bases for translation initiation. For hIAP, bIAP II, and bIAP IV, the second amino acid, that is, the one after the initiator methionine, is glutamine. Codons for glutamine all have a C in the first position. Thus, their Kozak sequences all have an ATGC (SEQ ID NO: 39) sequence. Accordingly, in various embodiments, the ATGC (SEQ ID NO: 39) sequence is changed to ATGG (SEQ ID NO: 40). This can be achieved by changing the second amino acid to a glycine, alanine, valine, aspartate, or glutamic acid, all of whose codons have a G in the first position. These amino acids may be compatible with signal peptide function. In alternative embodiments, the entire signal peptide is substituted for peptide having a canonical Kozak sequence and is derived from a highly expressed protein such as an immunoglobulin.

In various embodiments, the signal peptide of the AP-based agent may be deleted and/or substituted. For example, the signal peptide may be deleted, mutated, and/or substituted (e.g., with another signal peptide) to ensure protein expression.

In some embodiments, The DNA construct encoding the AP-based agent of the invention comprises untranslated DNA sequences. Such sequences include an intron, which may be heterologous to the IAP protein or native to the IAP protein including the native first and/or second intron and/or a native 3′ UTR. Without wishing to be bound by theory, it is believed that include of these sequences enhance protein expression by stabilizing the mRNA. Accordingly, in various embodiments, the DNA construct encoding the AP-based agent of the invention comprises the 5′UTR and/or the 3′UTR.

Provided below are illustrative IAP DNA sequences with a first intron and a 3′UTR:

hIAP with native first intron (shown as bolded and underlined)
SEQ ID NO: 12
ATGCAGGGGCCCTGGGTGCTGCTGCTGCTGGGCCTGAGGCTACAGCTCTCCCTGGGCG
TCATCCCAGGTAATGAGGCTCCCCAAGCTGTTCCACACACAGGGCACCCCCTCAGCCAG
GCTGACCTGATCTCTACTCTCCCCCTGGCCAGCTGAGGAGGAGAACCCGGCCTTCTGG
AACCGCCAGGCAGCTGAGGCCCTGGATGCTGCCAAGAAGCTGCAGCCCATCCAGAAGGT
CGCCAAGAACCTCATCCTCTTCCTGGGCGATGGGTTGGGGGTGCCCACGGTGACAGCCA
CCAGGATCCTAAAGGGGCAGAAGAATGGCAAACTGGGGCCTGAGACGCCCCTGGCCAT
GGACCGCTTCCCATACCTGGCTCTGTCCAAGACATACAATGTGGACAGACAGGTGCCAG
ACAGCGCAGCCACAGCCACGGCCTACCTGTGCGGGGTCAAGGCCAACTTCCAGACCATC
GGCTTGAGTGCAGCCGCCCGCTTTAACCAGTGCAACACGACACGCGGCAATGAGGTCAT
CTCCGTGATGAACCGGGCCAAGCAAGCAGGAAAGTCAGTAGGAGTGGTGACCACCACAC
GGGTGCAGCACGCCTCGCCAGCCGGCACCTACGCACACACAGTGAACCGCAACTGGTA
CTCAGATGCTGACATGCCTGCCTCAGCCCGCCAGGAGGGGTGCCAGGACATCGCCACTC
AGCTCATCTCCAACATGGACATTGACGTGATCCTTGGCGGAGGCCGCAAGTACATGTTTC
CCATGGGGACCCCAGACCCTGAGTACCCAGCTGATGCCAGCCAGAATGGAATCAGGCTG
GACGGGAAGAACCTGGTGCAGGAATGGCTGGCAAAGCACCAGGGTGCCTGGTATGTGT
GGAACCGCACTGAGCTCATGCAGGCGTCCCTGGACCAGTCTGTGACCCATCTCATGGGC
CTCTTTGAGCCCGGAGACACGAAATATGAGATCCACCGAGACCCCACACTGGACCCCTC
CCTGATGGAGATGACAGAGGCTGCCCTGCGCCTGCTGAGCAGGAACCCCCGCGGCTTCT
ACCTCTTTGTGGAGGGCGGCCGCATCGACCATGGTCATCATGAGGGTGTGGCTTACCAG
GCACTCACTGAGGCGGTCATGTTCGACGACGCCATTGAGAGGGCGGGCCAGCTCACCA
GCGAGGAGGACACGCTGACCCTCGTCACCGCTGACCACTCCCATGTCTTCTCCTTTGGT
GGCTACACCTTGCGAGGGAGCTCCATCTTCGGGTTGGCCCCCAGCAAGGCTCAGGACAG
CAAAGCCTACACGTCCATCCTGTACGGCAATGGCCCGGGCTACGTGTTCAACTCAGGCG
TGCGACCAGACGTGAATGAGAGCGAGAGCGGGAGCCCCGATTACCAGCAGCAGGCGGC
GGTGCCCCTGTCGTCCGAGACCCACGGAGGCGAAGACGTGGCGGTGTTTGCGCGCGGC
CCGCAGGCGCACCTGGTGCATGGTGTGCAGGAGCAGAGCTTCGTAGCGCATGTCATGG
CCTTCGCTGCCTGTCTGGAGCCCTACACGGCCTGCGACCTGGCGCCTCCCGCCTGCACC
ACCGACGCCGCGCACCCAGTTGCCGCGTCGCTGCCACTGCTGGCCGGGACCCTGCTGC
TGCTGGGGGCGTCCGCTGCTCCCTGA
hIAP with native 3′ UTR (shown as bolded and underlined)
SEQ ID NO: 13
ATGCAGGGGCCCTGGGTGCTGCTGCTGCTGGGCCTGAGGCTACAGCTCTCCCTGGGCG
TCATCCCAGCTGAGGAGGAGAACCCGGCCTTCTGGAACCGCCAGGCAGCTGAGGCCCT
GGATGCTGCCAAGAAGCTGCAGCCCATCCAGAAGGTCGCCAAGAACCTCATCCTCTTCCT
GGGCGATGGGTTGGGGGTGCCCACGGTGACAGCCACCAGGATCCTAAAGGGGCAGAAG
AATGGCAAACTGGGGCCTGAGACGCCCCTGGCCATGGACCGCTTCCCATACCTGGCTCT
GTCCAAGACATACAATGTGGACAGACAGGTGCCAGACAGCGCAGCCACAGCCACGGCCT
ACCTGTGCGGGGTCAAGGCCAACTTCCAGACCATCGGCTTGAGTGCAGCCGCCCGCTTT
AACCAGTGCAACACGACACGCGGCAATGAGGTCATCTCCGTGATGAACCGGGCCAAGCA
AGCAGGAAAGTCAGTAGGAGTGGTGACCACCACACGGGTGCAGCACGCCTCGCCAGCC
GGCACCTACGCACACACAGTGAACCGCAACTGGTACTCAGATGCTGACATGCCTGCCTC
AGCCCGCCAGGAGGGGTGCCAGGACATCGCCACTCAGCTCATCTCCAACATGGACATTG
ACGTGATCCTTGGCGGAGGCCGCAAGTACATGTTTCCCATGGGGACCCCAGACCCTGAG
TACCCAGCTGATGCCAGCCAGAATGGAATCAGGCTGGACGGGAAGAACCTGGTGCAGGA
ATGGCTGGCAAAGCACCAGGGTGCCTGGTATGTGTGGAACCGCACTGAGCTCATGCAGG
CGTCCCTGGACCAGTCTGTGACCCATCTCATGGGCCTCTTTGAGCCCGGAGACACGAAA
TATGAGATCCACCGAGACCCCACACTGGACCCCTCCCTGATGGAGATGACAGAGGCTGC
CCTGCGCCTGCTGAGCAGGAACCCCCGCGGCTTCTACCTCTTTGTGGAGGGCGGCCGC
ATCGACCATGGTCATCATGAGGGTGTGGCTTACCAGGCACTCACTGAGGCGGTCATGTTC
GACGACGCCATTGAGAGGGCGGGCCAGCTCACCAGCGAGGAGGACACGCTGACCCTCG
TCACCGCTGACCACTCCCATGTCTTCTCCTTTGGTGGCTACACCTTGCGAGGGAGCTCCA
TCTTCGGGTTGGCCCCCAGCAAGGCTCAGGACAGCAAAGCCTACACGTCCATCCTGTAC
GGCAATGGCCCGGGCTACGTGTTCAACTCAGGCGTGCGACCAGACGTGAATGAGAGCGA
GAGCGGGAGCCCCGATTACCAGCAGCAGGCGGCGGTGCCCCTGTCGTCCGAGACCCAC
GGAGGCGAAGACGTGGCGGTGTTTGCGCGCGGCCCGCAGGCGCACCTGGTGCATGGTG
TGCAGGAGCAGAGCTTCGTAGCGCATGTCATGGCCTTCGCTGCCTGTCTGGAGCCCTAC
ACGGCCTGCGACCTGGCGCCTCCCGCCTGCACCACCGACGCCGCGCACCCAGTTGCCG
CGTCGCTGCCACTGCTGGCCGGGACCCTGCTGCTGCTGGGGGCGTCCGCTGCTCCCTG
ATTTACTAAAACCTTGAAATAAAATTGTAAAACATCAGTTTGAAGGCCTGACTCTCAG
GGTAGTTCTTTTTTAATTCTGGGTTTT
bIAP IV with the first intron from bIAP I (shown as bolded
and underlined)
SEQ ID NO: 14
ATGCAGTGGGCCTGTGTGCTGCTGCTGCTGGGCCTGTGGCTACAGCTCTCCCTCACCTT
CATCCCAGGTAATCAGGCGGCTCCCAGCAGCCCCTACTCACAGGGGCGGCTCTAGGCT
GACCTGACCAACACTCTCCCCTTGGGCAGCTGAGGAGGAAGACCCCGCCTTCTGGAACC
GCCAGGCAGCCCAGGCCCTTGATGTAGCCAAGAAGTTGCAGCCGATCCAGACAGCTGCC
AAGAATGTCATCCTCTTCTTGGGGGATGGGATGGGGGTGCCTACGGTGACAGCCACTCG
GATCCTAAAGGGGCAGATGAATGGTAAGCTGGGACCTGAGACACCCCTGGCCATGGACC
AGTTCCCATACGTGGCTCTGTCCAAGACATACAACGTGGACAGACAGGTGCCAGACAGC
GCAGGCACTGCCACTGCCTACCTGTGTGGGGTCAAGGGCAACTACAAAACCATTGGTGT
AAGTGCAGCCGCCCGCTACAACCAGTGCAACACAACAAGTGGCAATGAGGTCACGTCTG
TGATGAACCGGGCCAAGAAAGCAGGAAAGTCAGTGGGAGTGGTGACCACCTCCAGGGTG
CAGCATGCCTCCCCAGCCGGTGCTTATGCACACACGGTGAACCGAAACTGGTACTCAGA
TGCCGACCTGCCTGCCGATGCACAGACGTATGGCTGCCAGGACATCGCCACACAACTGG
TCAACAACATGGATATTGACGTGATCCTGGGTGGAGGCCGAATGTACATGTTTCCTGAGG
GGACCCCGGATCCTGAATACCCATACGATGTCAATCAGACTGGAGTCCGGAAGGACAAG
CGGAATCTGGTGCAGGAGTGGCAGGCCAAGCACCAGGGAGCCCAGTATGTGTGGAACC
GCACGGAGCTCCTTCAGGCAGCCAATGACCCCAGTGTAACACACCTCATGGGCCTCTTT
GAGCCGGCAGACATGAAGTATAATGTTCAGCAAGACCCCACCAAGGACCCGACCCTGGA
GGAGATGACGGAGGCGGCCCTGCAAGTGCTGAGCAGGAACCCCCAGGGCTTCTACCTC
TTCGTGGAGGGAGGCCGCATTGACCACGGTCACCATGAAGGCAAAGCTTATATGGCACT
GACTGATACAGTCATGTTTGACAATGCCATCGCCAAGGCTAACGAGCTCACTAGCGAACT
GGACACGCTGATCCTTGCCACTGCAGACCACTCCCATGTCTTCTCTTTTGGTGGCTACAC
ACTGCGTGGGACCTCCATTTTCGGTCTGGCCCCCAGCAAGGCCTCAGACAACAAGTCCT
ACACCTCCATCCTCTATGGCAATGGCCCTGGCTACGTGCTTGGTGGGGGCTTAAGGCCC
GATGTTAATGACAGCATAAGCGAGGACCCCTCGTACCGGCAGCAGGCGGCCGTGCCCCT
GTCTAGTGAGTCCCACGGGGGCGAGGACGTGGCGGTGTTCGCGCGAGGCCCGCAGGC
GCACCTGGTGCACGGCGTGCAGGAGGAGACCTTCGTGGCGCACGTCATGGCCTTTGCG
GGCTGCGTGGAGCCCTACACCGACTGCAATCTGCCGGCCCCCTCTGGCCTCTCCGACGC
CGCGCACCTGGCGGCCAGCCCGCCTTCGCTGGCGCTGCTGGCCGGGGCGATGCTGCTG
CTGCTGGCGCCTGCCTTGTACTGA
bIAP IV with the 3′ UTR from bIAP I (shown as bolded and
underlined)
SEQ ID NO: 15
ATGCAGTGGGCCTGTGTGCTGCTGCTGCTGGGCCTGTGGCTACAGCTCTCCCTCACCTT
CATCCCAGCTGAGGAGGAAGACCCCGCCTTCTGGAACCGCCAGGCAGCCCAGGCCCTT
GATGTAGCCAAGAAGTTGCAGCCGATCCAGACAGCTGCCAAGAATGTCATCCTCTTCTTG
GGGGATGGGATGGGGGTGCCTACGGTGACAGCCACTCGGATCCTAAAGGGGCAGATGA
ATGGTAAGCTGGGACCTGAGACACCCCTGGCCATGGACCAGTTCCCATACGTGGCTCTG
TCCAAGACATACAACGTGGACAGACAGGTGCCAGACAGCGCAGGCACTGCCACTGCCTA
CCTGTGTGGGGTCAAGGGCAACTACAAAACCATTGGTGTAAGTGCAGCCGCCCGCTACA
ACCAGTGCAACACAACAAGTGGCAATGAGGTCACGTCTGTGATGAACCGGGCCAAGAAA
GCAGGAAAGTCAGTGGGAGTGGTGACCACCTCCAGGGTGCAGCATGCCTCCCCAGCCG
GTGCTTATGCACACACGGTGAACCGAAACTGGTACTCAGATGCCGACCTGCCTGCCGAT
GCACAGACGTATGGCTGCCAGGACATCGCCACACAACTGGTCAACAACATGGATATTGAC
GTGATCCTGGGTGGAGGCCGAATGTACATGTTTCCTGAGGGGACCCCGGATCCTGAATA
CCCATACGATGTCAATCAGACTGGAGTCCGGAAGGACAAGCGGAATCTGGTGCAGGAGT
GGCAGGCCAAGCACCAGGGAGCCCAGTATGTGTGGAACCGCACGGAGCTCCTTCAGGC
AGCCAATGACCCCAGTGTAACACACCTCATGGGCCTCTTTGAGCCGGCAGACATGAAGTA
TAATGTTCAGCAAGACCCCACCAAGGACCCGACCCTGGAGGAGATGACGGAGGCGGCC
CTGCAAGTGCTGAGCAGGAACCCCCAGGGCTTCTACCTCTTCGTGGAGGGAGGCCGCAT
TGACCACGGTCACCATGAAGGCAAAGCTTATATGGCACTGACTGATACAGTCATGTTTGA
CAATGCCATCGCCAAGGCTAACGAGCTCACTAGCGAACTGGACACGCTGATCCTTGCCA
CTGCAGACCACTCCCATGTCTTCTCTTTTGGTGGCTACACACTGCGTGGGACCTCCATTTT
CGGTCTGGCCCCCAGCAAGGCCTCAGACAACAAGTCCTACACCTCCATCCTCTATGGCAA
TGGCCCTGGCTACGTGCTTGGTGGGGGCTTAAGGCCCGATGTTAATGACAGCATAAGCG
AGGACCCCTCGTACCGGCAGCAGGCGGCCGTGCCCCTGTCTAGTGAGTCCCACGGGGG
CGAGGACGTGGCGGTGTTCGCGCGAGGCCCGCAGGCGCACCTGGTGCACGGCGTGCA
GGAGGAGACCTTCGTGGCGCACGTCATGGCCTTTGCGGGCTGCGTGGAGCCCTACACC
GACTGCAATCTGCCGGCCCCCTCTGGCCTCTCCGACGCCGCGCACCTGGCGGCCAGCC
CGCCTTCGCTGGCGCTGCTGGCCGGGGCGATGCTGCTGCTGCTGGCGCCTGCCTTGTA
CTGAGGGGACCCGGGGGTGGGGACACAGGCCCCGCCCTCCCTGGGAGGCAGGAAGCAG
CTCTCAAATAAACTGTTCTAAGTATGATACAGGAGTGATACATGTGTGAAGAGAAGCC
CTTAGGTGGGGGCACAGAGTGTCTGGGTGAGGGGGGTCAGGGTCACATCAGGAGGTTA
GGGAGGGGTTGATGAAGGGCTGACGTTGAGCAAAGACCAAAGGCAACTCAGAAGGACA
GTGGTGCAGGACTGGGTGTGGTCAGCAGGGGGCTGGTTGGGGGATCC

In various embodiments, the present invention contemplates the use of bacterial alkaline phosphatases. In some embodiments, the AP-based agent of the invention is derived from Bacillus subtilis. Bacillus subtilis is a Gram-positive bacterium found in soil and the gastrointestinal tract of humans. Bacillus subtilis secretes high levels of proteins into the environment and in the human GI tract that are properly folded. Without wishing to be bound by theory, it is believed that Bacillus subtilis secreted proteins in the GI tract may be resistant to degradation by common gastrointestinal proteases. Bacillus subtilis expresses at high levels an alkaline phosphatase multigene family. Among those isozymes, alkaline phosphatase IV is responsible for the majority of total alkaline phosphatase expression and activity in B. subtilis. In some embodiments, the AP-based agent of the invention is derived from Bacillus licheniformis. In some embodiments, the AP-based agent of the invention is derived from Escherichia coli.

Accordingly, in an illustrative embodiment, the AP-based agent of the invention is derived from alkaline phosphatase IV of Bacillus subtilis. In an embodiment, the bacterial alkaline phosphatase may have the following nucleotide and amino acid sequences:

Bacillus subtilis JH642 alkaline phosphatase IV,
mature protein nucleotide sequence
SEQ ID NO: 16
AAAAAACAAGACAAAGCTGAGATCAGAAATGTCATTGTGATGATAGGCGA
CGGCATGGGGACGCCTTACATAAGAGCCTACCGTTCCATGAAAAATAACG
GTGACACACCGAATAACCCGAAGTTAACAGAATTTGACCGGAACCTGACA
GGCATGATGATGACGCATCCGGATGACCCTGACTATAATATTACAGATTC
AGCAGCAGCCGGAACAGCATTAGCGACAGGCGTTAAGACATATAACAATG
CAATTGGCGTCGATAAAAACGGAAAAAAAGTGAAATCTGTACTTGAAGAG
GCCAAACAGCAAGGCAAGTCAACAGGGCTTGTCGCCACGTCTGAAATTAA
CCACGCCACTCCAGCCGCATATGGCGCCCACAATGAATCACGGAAAAACA
TGGACCAAATCGCCAACAGCTATATGGATGACAAGATAAAAGGCAAACAT
AAAATAGACGTGCTGCTCGGCGGCGGAAAATCTTATTTTAACCGCAAGAA
CAGAAACTTGACAAAGGAATTCAAACAAGCCGGCTACAGCTATGTGACAA
CTAAACAAGCATTGAAAAAAAATAAAGATCAGCAGGTGCTCGGGCTTTTC
GCAGATGGAGGGCTTGCTAAAGCGCTCGACCGTGACAGTAAAACACCGTC
TCTCAAAGACATGACGGTTTCAGCAATTGATCGCCTGAACCAAAATAAAA
AAGGATTTTTCTTGATGGTCGAAGGGAGCCAGATTGACTGGGCGGCCCAT
GACAATGATACAGTAGGAGCCATGAGCGAGGTTAAAGATTTTGAACAGGC
CTATAAAGCCGCGATTGAATTTGCGAAAAAAGACAAACATACACTTGTGA
TTGCAACTGCTGACCATACAACCGGCGGCTTTACCATTGGCGCAAACGGG
GAAAAGAATTGGCACGCAGAACCGATTCTCTCCGCTAAGAAAACACCTGA
ATTCATGGCCAAAAAAATCAGTGAAGGCAAGCCGGTTAAAGATGTGCTCG
CCCGCTATGCCAATCTGAAAGTCACATCTGAAGAAATCAAAAGCGTTGAA
GCAGCTGCACAGGCTGACAAAAGCAAAGGGGCCTCCAAAGCCATCATCAA
GATTTTTAATACCCGCTCCAACAGCGGATGGACGAGTACCGATCATACCG
GCGAAGAAGTACCGGTATACGCGTACGGCCCCGGAAAAGAAAAATTCCGC
GGATTGATTAACAATACGGACCAGGCAAACATCATATTTAAGATTTTAAA
AACTGGAAAA
Bacillus subtilis JH642 alkaline phosphatase IV,
mature protein amino acid sequence
SEQ ID NO: 17
KKQDKAEIRNVIVMIGDGMGTPYIRAYRSMKNNGDTPNNPKLTEFDRNLT
GMMMTHPDDPDYNITDSAAAGTALATGVKTYNNAIGVDKNGKKVKSVLEE
AKQQGKSTGLVATSEINHATPAAYGAHNESRKNMDQIANSYMDDKIKGKH
KIDVLLGGGKSYFNRKNRNLTKEFKQAGYSYVTTKQALKKNKDQQVLGLF
ADGGLAKALDRDSKTPSLKDMTVSAIDRLNQNKKGFFLMVEGSQIDWAAH
DNDTVGAMSEVKDFEQAYKAAIEFAKKDKHTLVIATADHTTGGFTIGANG
EKNWHAEPILSAKKTPEFMAKKISEGKPVKDVLARYANLKVTSEEIKSVE
AAAQADKSKGASKAIIKIFNTRSNSGWTSTDHTGEEVPVYAYGPGKEKFR
GLINNTDQANIIFKILKTGK

In some embodiments, the AP-based agent includes bacterial alkaline phosphatases that has one or more mutations that alter catalytic activity. In some embodiments, the bacterial alkaline phosphatases include one or more mutations such that their catalytic activity is similar or higher than mammalian alkaline phosphatases. In some embodiments, the bacterial alkaline phosphatases include one or more mutations that alter their de-phosphorylation profile. In an embodiment, the bacterial alkaline phosphatases of the invention exhibits similar de-phosphorylation profile as mammalian alkaline phosphatases. In some embodiments, the bacterial alkaline phosphatases include one or more mutations that alter their activity at higher pH. In an embodiment, the bacterial alkaline phosphatases of the invention exhibits similar activity at higher pH as mammalian alkaline phosphatases. In some embodiments, the bacterial alkaline phosphatases include one or more mutations that alter their metal requirements. In an embodiment, the bacterial alkaline phosphatases of the invention exhibits metal requirements (e.g., Mg) as mammalian alkaline phosphatases.

For example, in certain embodiments, the AP-based agent of the invention is derived from Bacillus subtilis JH642 alkaline phosphatase IV, and has one or more mutations at positions 101, 328, A330, and 374. For example, the AP-based agent may include one or more of the following mutations: D101A, W328H, A330N and G374C.

In various embodiments, the AP-based agent of the invention comprises a nucleotide sequence having at least about 60% (e.g. about 60%, or about 61%, or about 62%, or about 63%, or about 64%, or about 65%, or about 66%, or about 67%, or about 68%, or about 69%, or about 70%, or about 71%, or about 72%, or about 73%, or about 74%, or about 75%, or about 76%, or about 77%, or about 78%, or about 79%, or about 80%, or about 81%, or about 82%, or about 83%, or about 84%, or about 85%, or about 86%, or about 87%, or about 88%, or about 89%, or about 90%, or about 91%, or about 92%, or about 93%, or about 94%, or about 95%, or about 96%, or about 97%, or about 98%, or about 99%) sequence identity with any of the sequences disclosed herein.

In some embodiments, the AP-based agent of the invention comprises an amino sequence having at least about 60% (e.g. about 60%, or about 61%, or about 62%, or about 63%, or about 64%, or about 65%, or about 66%, or about 67%, or about 68%, or about 69%, or about 70%, or about 71%, or about 72%, or about 73%, or about 74%, or about 75%, or about 76%, or about 77%, or about 78%, or about 79%, or about 80%, or about 81%, or about 82%, or about 83%, or about 84%, or about 85%, or about 86%, or about 87%, or about 88%, or about 89%, or about 90%, or about 91%, or about 92%, or about 93%, or about 94%, or about 95%, or about 96%, or about 97%, or about 98%, or about 99%) sequence identity with any of the sequences disclosed herein.

In various embodiments, the AP-based agent of the invention may comprise an amino acid sequence having one or more amino acid mutations relative any of the protein sequences described herein. In some embodiments, the one or more amino acid mutations may be independently selected from substitutions, insertions, deletions, and truncations.

In some embodiments, the amino acid mutations are amino acid substitutions, and may include conservative and/or non-conservative substitutions.

“Conservative substitutions” may be made, for instance, on the basis of similarity in polarity, charge, size, solubility, hydrophobicity, hydrophilicity, and/or the amphipathic nature of the amino acid residues involved. The 20 naturally occurring amino acids can be grouped into the following six standard amino acid groups: (1) hydrophobic: Met, Ala, Val, Leu, lle; (2) neutral hydrophilic: Cys, Ser, Thr; Asn, GIn; (3) acidic: Asp, Glu; (4) basic: His, Lys, Arg; (5) residues that influence chain orientation: Gly, Pro; and (6) aromatic: Trp, Tyr, Phe.

As used herein, “conservative substitutions” are defined as exchanges of an amino acid by another amino acid listed within the same group of the six standard amino acid groups shown above. For example, the exchange of Asp by Glu retains one negative charge in the so modified polypeptide. In addition, glycine and proline may be substituted for one another based on their ability to disrupt α-helices.

As used herein, “non-conservative substitutions” are defined as exchanges of an amino acid by another amino acid listed in a different group of the six standard amino acid groups (1) to (6) shown above.

In various embodiments, the substitutions may also include non-classical amino acids (e.g. selenocysteine, pyrrolysine, N-formylmethionine β-alanine, GABA and δ-Aminolevulinic acid, 4-aminobenzoic acid (PABA), D-isomers of the common amino acids, 2,4-diaminobutyric acid, α-amino isobutyric acid, 4-aminobutyric acid, Abu, 2-amino butyric acid, γ-Abu, ε-Ahx, 6-amino hexanoic acid, Aib, 2-amino isobutyric acid, 3-amino propionic acid, ornithine, norleucine, norvaline, hydroxyproline, sarcosme, citrulline, homocitrulline, cysteic acid, t-butylglycine, t-butylalanine, phenylglycine, cyclohexylalanine, β-alanine, fluoro-amino acids, designer amino acids such as β methyl amino acids, C α-methyl amino acids, N α-methyl amino acids, and amino acid analogs in general).

Mutations may also be made to the nucleotide sequences of the alkaline phosphatases by reference to the genetic code, including taking into account codon degeneracy. In various embodiments, the DNA construct encoding the AP-based agent is codon optimized for protein expression in the host cell.

Mutations may be made to the AP-based agent of the invention to select for agents with desired characteristics. For examples, mutations may be made to generate AP-based agents with enhanced catalytic activity or protein stability. In various embodiments, directed evolution may be utilized to generate AP-based agents of the invention. For example, error-prone PCR and DNA shuffling may be used to identify mutations in the bacterial alkaline phosphatases that confer enhanced activity.

In various embodiments, the AP-based agent of the invention possesses desirable characteristics, including, for example, high specific activity. In various embodiments, the alkaline phosphatase of the invention possesses a specific activity of at least about 100 U/mg to about 20,000 U/mg. In various embodiments, the alkaline phosphatase of the invention possesses a specific activity of at least about 100 U/mg, about 200 U/mg, about 300 U/mg, about 400 U/mg, about 500 U/mg, about 600 U/mg, about 700 U/mg, about 800 U/mg, about 900 U/mg, about 1,000 U/mg, about 2,000 U/mg, about 3,000 U/mg, about 4,000 U/mg, about 5,000 U/mg, about 6,000 U/mg, about 7,000 U/mg, about 8,000 U/mg, about 9,000 U/mg, about 10,000 U/mg, about 11,000 U/mg, about 12,000 U/mg, about 13,000 U/mg, about 14,000 U/mg, about 15,000 U/mg, about 16,000 U/mg, about 17,000 U/mg, about 18,000 U/mg, about 19,000 U/mg, or about 20,000 U/mg.

In various embodiments, the AP-based agent of the invention is stable and/or active in the GI tract, e.g. in one or more of the mouth, esophagus, stomach, duodenum, small intestine, duodenum, jejunum, ileum, large intestine, colon transversum, colon descendens, colon ascendens, colon sigmoidenum, cecum, and rectum. In a specific embodiment, the alkaline phosphatase is stable in the large intestine, optionally selected from one or more of colon transversum, colon descendens, colon ascendens, colon sigmoidenum and cecum. In a specific embodiment, the alkaline phosphatase is stable in the small intestine, optionally selected from one or more of duodenum, jejunum, and ileum. In some embodiments, the alkaline phosphatase is resistant to proteases in the GI tract, including for example, the small intestine. In some embodiments, the alkaline phosphatase is substantially active at a pH of about 5.0 or above. For example, the alkaline phosphatase may be substantially active at a pH about 6.0 to about 12, e.g. about 6.0, or about 6.1, or about 6.2, or about 6.3, or about 6.4, or about 6.5, or about 6.6, or about 6.7, or about 6.8, or about 6.9, or about 7.0, or about 7.1, or about 7.2, or about 7.3, or about 7.4, or about 7.5, or about 8.0, or about 8.5, or about 9.0, or about 9.5, or about 10.0, or about 10.5, or about 11.0, or about 11.5, or about 12.0 (including, for example, via formulation, as described herein). In some embodiments, stable refers to an enzyme that has a long enough half-life and maintains sufficient activity for therapeutic effectiveness.

In various embodiments, the AP-based agent of the invention is stable in chyme.

In some embodiments, the AP-based agent described herein, include derivatives that are modified, i.e., by the covalent attachment of any type of molecule to the alkaline phosphatase such that covalent attachment does not prevent the activity of the enzyme. For example, but not by way of limitation, derivatives include alkaline phosphatases that have been modified by, inter alia, glycosylation, lipidation, acetylation, pegylation, phosphorylation, amidation, derivatization by known protecting/blocking groups, proteolytic cleavage, linkage to a cellular ligand or other protein, etc. Any of numerous chemical modifications can be carried out, including, but not limited to specific chemical cleavage, acetylation, formylation, metabolic synthesis of tunicamycin, etc. Additionally, the derivative can contain one or more non-classical amino acids. In various embodiments, the AP-based agent is glycosylated to ensure proper protein folding.

In still other embodiments, the AP-based agents of the invention may be modified to add effector moieties such as chemical linkers, detectable moieties such as for example fluorescent dyes, enzymes, substrates, bioluminescent materials, radioactive materials, and chemiluminescent moieties, or functional moieties such as for example streptavidin, avidin, biotin, a cytotoxin, a cytotoxic agent, and radioactive materials. The AP-based agent described herein can possess a sufficiently basic functional group, which can react with an inorganic or organic acid, or a carboxyl group, which can react with an inorganic or organic base, to form a pharmaceutically acceptable salt. A pharmaceutically acceptable acid addition salt is formed from a pharmaceutically acceptable acid, as is well known in the art. Such salts include the pharmaceutically acceptable salts listed in, for example, Journal of Pharmaceutical Science, 66, 2-19 (1977) and The Handbook of Pharmaceutical Salts; Properties, Selection, and Use. P. H. Stahl and C. G. Wermuth (eds.), Verlag, Zurich (Switzerland) 2002, which are hereby incorporated by reference in their entirety.

Pharmaceutically acceptable salts include, by way of non-limiting example, sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, pamoate, phenylacetate, trifluoroacetate, acrylate, chlorobenzoate, dinitrobenzoate, hydroxybenzoate, methoxybenzoate, methylbenzoate, o-acetoxybenzoate, naphthalene-2-benzoate, isobutyrate, phenylbutyrate, α-hydroxybutyrate, butyne-1,4-dicarboxylate, hexyne-1,4-dicarboxylate, caprate, caprylate, cinnamate, glycollate, heptanoate, hippurate, malate, hydroxymaleate, malonate, mandelate, mesylate, nicotinate, phthalate, teraphthalate, propiolate, propionate, phenylpropionate, sebacate, suberate, p-bromobenzenesulfonate, chlorobenzenesulfonate, ethylsulfonate, 2-hydroxyethylsulfonate, methylsulfonate, naphthalene-1-sulfonate, naphthalene-2-sulfonate, naphthalene-1,5-sulfonate, xylenesulfonate, and tartarate salts.

The term “pharmaceutically acceptable salt” also refers to a salt of the alkaline phosphatases having an acidic functional group, such as a carboxylic acid functional group, and a base. Suitable bases include, but are not limited to, hydroxides of alkali metals such as sodium, potassium, and lithium; hydroxides of alkaline earth metal such as calcium and magnesium; hydroxides of other metals, such as aluminum and zinc; ammonia, and organic amines, such as unsubstituted or hydroxy-substituted mono-, di-, or tri-alkylamines, dicyclohexylamine; tributyl amine; pyridine; N-methyl, N-ethylamine; diethylamine; triethylamine; mono-, bis-, or tris-(2-OH-lower alkylamines), such as mono-; bis-, or tris-(2-hydroxyethyl) amine, 2-hydroxy-tert-butylamine, or tris-(hydroxymethyl) methylamine, N,N-di-lower alkyl-N-(hydroxyl-lower alkyl)-amines, such as N,N-dimethyl-N-(2-hydroxyethyl) amine or tri-(2-hydroxyethyl) amine; N-methyl-D-glucamine; and amino acids such as arginine, lysine, and the like.

In some embodiments, the compositions described herein are in the form of a pharmaceutically acceptable salt.

Further, any AP-based agent described herein can be administered to a subject as a component of a composition that comprises a pharmaceutically acceptable carrier or vehicle. Such compositions can optionally comprise a suitable amount of a pharmaceutically acceptable excipient so as to provide the form for proper administration.

Pharmaceutical excipients can be liquids, such as water and oils, including those of petroleum, animal, vegetable, or synthetic origin, such as peanut oil, soybean oil, mineral oil, sesame oil and the like. The pharmaceutical excipients can be, for example, saline, gum acacia, gelatin, starch paste, talc, keratin, colloidal silica, urea and the like. In addition, auxiliary, stabilizing, thickening, lubricating, and coloring agents can be used. In one embodiment, the pharmaceutically acceptable excipients are sterile when administered to a subject. Water is a useful excipient when any agent described herein is administered intravenously. Saline solutions and aqueous dextrose and glycerol solutions can also be employed as liquid excipients, specifically for injectable solutions. Suitable pharmaceutical excipients also include starch, glucose, cellulose, hypromellose, lactose, sucrose, malt, rice, flour, chalk, silica gel, sodium stearate, glycerol monostearate, talc, sodium chloride, dried skim milk, glycerol, propylene, glycol, povidone, crosspovidone, water, ethanol and the like. Any agent described herein, if desired, can also comprise minor amounts of wetting or emulsifying agents, or pH buffering agents. Other examples of suitable pharmaceutical excipients are described in Remington's Pharmaceutical Sciences 1447-1676 (Alfonso R. Gennaro eds., 19th ed. 1995), incorporated herein by reference.

Where necessary, the AP-based agent and/or pharmaceutical compositions (and/or additional therapeutic agents) can include a solubilizing agent. Also, the agents can be delivered with a suitable vehicle or delivery device. Compositions for administration can optionally include a local anesthetic such as, for example, lignocaine to lessen pain at the site of the injection. Combination therapies outlined herein can be co-delivered in a single delivery vehicle or delivery device.

### Methods of Making IAP

The IAPs of the invention are made using standard molecular biology techniques. For example, nucleic acid compositions encoding the IAPs of the invention are also provided, as well as expression vectors containing the nucleic acids and host cells transformed with the nucleic acid and/or expression vector compositions. As will be appreciated by those in the art, the protein sequences depicted herein can be encoded by any number of possible nucleic acid sequences, due to the degeneracy of the genetic code.

As is known in the art, the nucleic acids encoding the components of the invention can be incorporated into expression vectors as is known in the art, and depending on the host cells, used to produce the IAP compositions of the invention. Generally, the nucleic acids are operably linked to any number of regulatory elements (promoters, origin of replication, selectable markers, ribosomal binding sites, inducers, etc.). The expression vectors can be extra-chromosomal or integrating vectors.

The nucleic acids and/or expression vectors of the invention are then transformed into any number of different types of host cells as is well known in the art, including mammalian, bacterial, yeast, insect and/or fungal cells, with mammalian cells (e.g. CHO cells), finding use in many embodiments.

The IAPs of the invention are made by culturing host cells comprising the expression vector(s) as is well known in the art. Once produced, traditional purification steps are done.

### Formulations Comprising AP-Based Agents

The present invention provides the described AP-based agent and/or pharmaceutical compositions (and/or additional therapeutic agents) in various formulations. Any AP-based agent and/or pharmaceutical composition (and/or additional therapeutic agents) described herein can take the form of tablets, pills, pellets, capsules, capsules containing liquids, capsules containing multiparticulates, powders, solutions, emulsion, drops, suppositories, emulsions, aerosols, sprays, suspensions, delayed-release formulations, sustained-release formulations, controlled-release formulations, or any other form suitable for use.

The formulations comprising the AP-based agent and/or pharmaceutical compositions (and/or additional therapeutic agents) may conveniently be presented in unit dosage forms. For example, the dosage forms may be prepared by methods which include the step of bringing the therapeutic agents into association with a carrier, which constitutes one or more accessory ingredients. For example, the formulations are prepared by uniformly and intimately bringing the therapeutic agent into association with a liquid carrier, a finely divided solid carrier, or both, and then, if necessary, shaping the product into dosage forms of the desired formulation (e.g., wet or dry granulation, powder blends, etc., followed by press tableting)

In one embodiment, an IAP (and/or additional therapeutic agents) described herein is formulated as a composition adapted for a mode of administration described herein.

In some embodiments, the administration an IAP and/or pharmaceutical compositions (and/or additional therapeutic agents) is any one of oral, intravenous, and parenteral. For example, routes of administration include, but are not limited to, oral, intradermal, intramuscular, intraperitoneal, intravenous, subcutaneous, intranasal, epidural, sublingual, intranasal, intracerebral, intravaginal, transdermal, rectally, by inhalation, or topically (e.g., to the ears, nose, eyes, or skin).

In some embodiments, an IAP and/or pharmaceutical compositions (and/or additional therapeutic agents) described herein is formulated as a composition adapted for oral administration. Compositions for oral delivery can be in the form of tablets, lozenges, aqueous or oily suspensions, granules, powders, sprinkles, emulsions, capsules, syrups, or elixirs, for example. Orally administered compositions can comprise one or more agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions can be coated to delay disintegration to provide a sustained action over an extended period of time. Selectively permeable membranes surrounding an osmotically active agent driving any alkaline phosphatase (and/or additional therapeutic agents) described herein are also suitable for orally administered compositions. In these latter platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time-delay material such as glycerol monostearate or glycerol stearate can also be useful. Oral compositions can include standard excipients such as mannitol, lactose, starch, magnesium stearate, sodium saccharin, cellulose, ethacrylic acid and derivative polymers thereof, and magnesium carbonate. In one embodiment, the excipients are of pharmaceutical grade. Suspensions, in addition to the active compounds, may contain suspending agents such as, for example, ethoxylated isostearyl alcohols, polyoxyethylene sorbitol and sorbitan esters, microcrystalline cellulose, aluminum metahydroxide, bentonite, agar-agar, tragacanth, etc., and mixtures thereof.

In various embodiments, the AP-based agent and/or pharmaceutical compositions (and/or additional therapeutic agent) are formulated as solid dosage forms such as tablets, dispersible powders, granules, and capsules. In one embodiment, the AP-based agent and/or pharmaceutical compositions (and/or additional therapeutic agent) are formulated as a capsule. In another embodiment, the AP-based agent and/or pharmaceutical compositions (and/or additional therapeutic agent) are formulated as a tablet. In yet another embodiment, the AP-based agent and/or pharmaceutical compositions (and/or additional therapeutic agent) are formulated as a soft-gel capsule. In a further embodiment, the AP-based agent and/or pharmaceutical compositions (and/or additional therapeutic agent) are formulated as a gelatin capsule.

Dosage forms suitable for parenteral administration (e.g. intravenous, intramuscular, intraperitoneal, subcutaneous and intra-articular injection and infusion) include, for example, solutions, suspensions, dispersions, emulsions, and the like. They may also be manufactured in the form of sterile solid compositions (e.g., lyophilized composition), which can be dissolved or suspended in sterile injectable medium immediately before use. They may contain, for example, suspending or dispersing agents.

In various embodiments, the formulations of the AP-based agents, such as an IAP, may additionally comprise a pharmaceutically acceptable carrier or excipient. As one skilled in the art will recognize, the formulations can be in any suitable form appropriate for the desired use and route of administration.

In some dosage forms, the agents described herein are mixed with at least one inert, pharmaceutically acceptable excipient or carrier such as sodium citrate, dicalcium phosphate, etc., and/or a) fillers or extenders such as starches, lactose, sucrose, glucose, mannitol, silicic acid, microcrystalline cellulose, and Bakers Special Sugar, etc., b) binders such as, for example, carboxymethylcellulose, alginates, gelatin, polyvinylpyrrolidone, sucrose, acacia, polyvinyl alcohol, polyvinylpyrrolidone, methylcellulose, hydroxypropyl cellulose (HPC), and hydroxymethyl cellulose etc., c) humectants such as glycerol, etc., d) disintegrating agents such as agar-agar, calcium carbonate, potato or tapioca starch, alginic acid, certain silicates, sodium carbonate, cross-linked polymers such as crospovidone (cross-linked polyvinylpyrrolidone), croscarmellose sodium (cross-linked sodium carboxymethylcellulose), sodium starch glycolate, etc., e) solution retarding agents such as paraffin, etc., f) absorption accelerators such as quaternary ammonium compounds, etc., g) wetting agents such as, for example, cetyl alcohol and glycerol monostearate, etc., h) absorbents such as kaolin and bentonite clay, etc., and i) lubricants such as talc, calcium stearate, magnesium stearate, solid polyethylene glycols, sodium lauryl sulfate, glyceryl behenate, etc., and mixtures of such excipients. One of skill in the art will recognize that particular excipients may have two or more functions in the oral dosage form. In the case of an oral dosage form, for example, a capsule or a tablet, the dosage form may also comprise buffering agents.

The formulation can additionally include a surface active agent. Surface active agents suitable for use in the present invention include, but are not limited to, any pharmaceutically acceptable, non-toxic surfactant. Classes of surfactants suitable for use in the compositions of the invention include, but are not limited to polyethoxylated fatty acids, PEG-fatty acid diesters, PEG-fatty acid mono-and di-ester mixtures, polyethylene glycol glycerol fatty acid esters, alcohol-oil transesterification products, polyglycerized fatty acids, propylene glycol fatty acid esters, mixtures of propylene glycol esters-glycerol esters, mono-and diglycerides, sterol and sterol derivatives, polyethylene glycol sorbitan fatty acid esters, polyethylene glycol alkyl ethers, sugar esters, polyethylene glycol alkyl phenols, polyoxyethylene-olyoxypropylene block copolymers, sorbitan fatty acid esters, lower alcohol fatty acid esters, ionic surfactants, and mixtures thereof. In some embodiments, compositions of the invention may comprise one or more surfactants including, but not limited to, sodium lauryl sulfate, polysorbate 20, polysorbate 40, polysorbate 60, polysorbate 80, and triethyl citrate.

The formulation can also contain pharmaceutically acceptable plasticizers to obtain the desired mechanical properties such as flexibility and hardness. Such plasticizers include, but are not limited to, triacetin, citric acid esters, triethyl citrate, phthalic acid esters, dibutyl sebacate, cetyl alcohol, polyethylene glycols, polysorbates or other plasticizers.

The formulation can also include one or more application solvents. Some of the more common solvents that can be used to apply, for example, a delayed-release coating composition include isopropyl alcohol, acetone, methylene chloride and the like.

The formulation can also include one or more alkaline materials. Alkaline material suitable for use in compositions of the invention include, but are not limited to, sodium, potassium, calcium, magnesium and aluminum salts of acids such as phosphoric acid, carbonic acid, citric acid and other aluminum/magnesium compounds. In addition, the alkaline material may be selected from antacid materials such as aluminum hydroxides, calcium hydroxides, magnesium hydroxides and magnesium oxide.

In various embodiments, the formulation can additionally include magnesium and/or zinc. The inclusion of magnesium and/or zinc in the formulation may promote protein folding (e.g., dimer formation) and bioactivity of the AP-based agent. In some embodiments, the formulation can include magnesium at a concentration of from about 1 μM to greater than 500 mM (e.g., from about 1 μM to more than 5 mM), inclusive of all ranges and values therebetween. In some embodiments, the formulation can include zinc at a concentration of about 1 μM to greater than 100 mM (e.g., from about 1 μM to more than 1 mM), inclusive of all ranges and values therebetween. In various embodiments, the formulation of the present invention is substantially free of metal chelators.

In various embodiments, the pH of the formulation ensures that the AP-based agent is properly folded (e.g., dimer formation) and is bioactive. In some embodiments, the formulation is maintained at a pH such that the amino acids which coordinate the binding of magnesium and/or zinc within the AP-based agent are not protonated. Protonation of such coordinating amino acids may lead to loss of metal ions and bioactivity and dimer disassociation. In various embodiments, the pH of the formulation is greater than about 6, about 6.5, about 7, about 7.5, about 8, about 8.5, about 9, about 9.5, about 10, about 10.5, about 11, about 11.5, or about 12.

Besides inert diluents, the oral compositions can also include adjuvants such as sweetening, flavoring, and perfuming agents.

In various embodiments, the AP-based agent and/or pharmaceutical compositions (and/or additional therapeutic agents) are formulated for systemic or local delivery. In an embodiment, administration is systemic. In another embodiment, it may be desirable to administer locally to the area in need of treatment.

Various methods may be used to formulate and/or deliver the agents described herein to a location of interest. For example, the intestinal alkaline phosphatase and/or pharmaceutical compositions (and/or additional therapeutic agents) described herein may be formulated for delivery to the gastrointestinal tract. The gastrointestinal tract includes organs of the digestive system such as mouth, esophagus, stomach, duodenum, small intestine, large intestine and rectum and includes all subsections thereof (e.g., the small intestine may include the duodenum, jejunum and ileum; the large intestine may include the colon transversum, colon descendens, colon ascendens, colon sigmoidenum and cecum). For example, the alkaline phosphatases and/or pharmaceutical compositions (and/or additional therapeutic agents) described herein may be formulated for delivery to one or more of the stomach, small intestine, large intestine and rectum and includes all subsections thereof (e.g., duodenum, jejunum and ileum, colon transversum, colon descendens, colon ascendens, colon sigmoidenum and cecum). In some embodiments, the compositions described herein may be formulated to deliver to the upper or lower GI tract. In an embodiment, the alkaline phosphatases and/or pharmaceutical compositions (and/or additional therapeutic agents) may be administered to a subject, by, for example, directly or indirectly contacting the mucosal tissues of the gastrointestinal tract.

In various embodiments, the administration the AP-based agent and/or pharmaceutical compositions (and/or additional therapeutic agents) is into the GI tract via, for example, oral delivery, nasogastral tube, intestinal intubation (e.g., an enteral tube or feeding tube such as, for example, a jejunal tube or gastro-jejunal tube, etc.), direct infusion (e.g., duodenal infusion), endoscopy, colonoscopy, or enema.

### Administration and Dosages

As a person of skill in the art will appreciate, the actual dose of an IAP to be administered according to the present invention will vary according to the particular compound, the particular dosage form, and the mode of administration. Many factors that may modify the action of the IAP (e.g., body weight, gender, diet, time of administration, route of administration, rate of excretion, condition of the subject, drug combinations, genetic disposition and reaction sensitivities) can be taken into account by those skilled in the art. Administration can be carried out continuously or in one or more discrete doses within the maximum tolerated dose. Administration rates for a given set of conditions can be ascertained by those skilled in the art using conventional dosage administration tests.

Individual doses of the AP-based agent, such as the IAP, can be administered in unit dosage forms (e.g., tablets or capsules) containing, for example, from about 0.01 mg to about 1,000 mg, about 0.01 mg to about 900 mg, about 0.01 mg to about 800 mg, about 0.01 mg to about 700 mg, about 0.01 mg to about 600 mg, about 0.01 mg to about 500 mg, about 0.01 mg to about 400 mg, about 0.01 mg to about 300 mg, about 0.01 mg to about 200 mg, from about 0.1 mg to about 100 mg, from about 0.1 mg to about 90 mg, from about 0.1 mg to about 80 mg, from about 0.1 mg to about 70 mg, from about 0.1 mg to about 60 mg, from about 0.1 mg to about 50 mg, from about 0.1 mg to about 40 mg, from about 0.1 mg to about 30 mg, from about 0.1 mg to about 20 mg, from about 0.1 mg to about 10 mg, from about 0.1 mg to about 5 mg, from about 0.1 mg to about 3 mg, or from about 0.1 mg to about 1 mg active ingredient per unit dosage for. For example, a unit dosage form can be about 0.01 mg, about 0.02 mg, about 0.03 mg, about 0.04 mg, about 0.05 mg, about 0.06 mg, about 0.07 mg, about 0.08 mg, about 0.09 mg, about 0.1 mg, about 0.2 mg, about 0.3 mg, about 0.4 mg, about 0.5 mg, about 0.6 mg, about 0.7 mg, about 0.8 mg, about 0.9 mg, about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 61 mg, about 62 mg, about 63 mg, about 64 mg, about 65 mg, about 66 mg, about 67 mg, about 68 mg, about 69 mg, about 70 mg, about 71 mg, about 72 mg, about 73 mg, about 74 mg, about 75 mg, about 76 mg, about 77 mg, about 78 mg, about 79 mg, about 80 mg, about 81 mg, about 82 mg, about 83 mg, about 84 mg, about 85 mg, about 86 mg, about 87 mg, about 88 mg, about 89 mg, about 90 mg, about 91 mg, about 92 mg, about 93 mg, about 94 mg, about 95 mg, about 96 mg, about 97 mg, about 98 mg, about 99 mg, about 100 mg, about 200 mg, about 300 mg, about 400 mg, about 500 mg, about 600 mg, about 700 mg, about 800 mg, about 900 mg, or about 1,000 mg of the AP-based agent, inclusive of all values and ranges therebetween.

In one embodiment, the AP-based agent, such as the IAP, is administered at an amount of from about 0.01 mg to about 1,000 mg daily, about 0.01 mg to about 900 mg daily, about 0.01 mg to about 800 mg daily, about 0.01 mg to about 700 mg daily, about 0.01 mg to about 600 mg daily, about 0.01 mg to about 500 mg daily, about 0.01 mg to about 400 mg daily, about 0.01 mg to about 300 mg daily, about 0.01 mg to about 200 mg daily, about 0.01 mg to about 100 mg daily, an amount of from about 0.1 mg to about 100 mg daily, from about 0.1 mg to about 95 mg daily, from about 0.1 mg to about 90 mg daily, from about 0.1 mg to about 85 mg daily, from about 0.1 mg to about 80 mg daily, from about 0.1 mg to about 75 mg daily, from about 0.1 mg to about 70 mg daily, from about 0.1 mg to about 65 mg daily, from about 0.1 mg to about 60 mg daily, from about 0.1 mg to about 55 mg daily, from about 0.1 mg to about 50 mg daily, from about 0.1 mg to about 45 mg daily, from about 0.1 mg to about 40 mg daily, from about 0.1 mg to about 35 mg daily, from about 0.1 mg to about 30 mg daily, from about 0.1 mg to about 25 mg daily, from about 0.1 mg to about 20 mg daily, from about 0.1 mg to about 15 mg daily, from about 0.1 mg to about 10 mg daily, from about 0.1 mg to about 5 mg daily, from about 0.1 mg to about 3 mg daily, from about 0.1 mg to about 1 mg daily, or from about 5 mg to about 80 mg daily. In various embodiments, the AP-based agent is administered at a daily dose of about 0.01 mg, about 0.02 mg, about 0.03 mg, about 0.04 mg, about 0.05 mg, about 0.06 mg, about 0.07 mg, about 0.08 mg, about 0.09 mg, about 0.1 mg, about 0.2 mg, about 0.3 mg, about 0.4 mg, about 0.5 mg, about 0.6 mg, about 0.7 mg, about 0.8 mg, about 0.9 mg, about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 61 mg, about 62 mg, about 63 mg, about 64 mg, about 65 mg, about 66 mg, about 67 mg, about 68 mg, about 69 mg, about 70 mg, about 71 mg, about 72 mg, about 73 mg, about 74 mg, about 75 mg, about 76 mg, about 77 mg, about 78 mg, about 79 mg, about 80 mg, about 81 mg, about 82 mg, about 83 mg, about 84 mg, about 85 mg, about 86 mg, about 87 mg, about 88 mg, about 89 mg, about 90 mg, about 91 mg, about 92 mg, about 93 mg, about 94 mg, about 95 mg, about 96 mg, about 97 mg, about 98 mg, about 99 mg, about 100 mg, about 200 mg, about 300 mg, about 400 mg, about 500 mg, about 600 mg, about 700 mg, about 800 mg, about 900 mg, or about 1,000 mg, inclusive of all values and ranges therebetween.

In some embodiments, a suitable dosage of the AP-based agent is in a range of about 0.01 mg/kg to about 100 mg/kg of body weight of the subject, about 0.01 mg/kg to about 90 mg/kg of body weight of the subject, about 0.01 mg/kg to about 80 mg/kg of body weight of the subject, about 0.01 mg/kg to about 70 mg/kg of body weight of the subject, about 0.01 mg/kg to about 60 mg/kg of body weight of the subject, about 0.01 mg/kg to about 50 mg/kg of body weight of the subject, about 0.01 mg/kg to about 40 mg/kg of body weight of the subject, about 0.01 mg/kg to about 30 mg/kg of body weight of the subject, about 0.01 mg/kg to about 20 mg/kg of body weight of the subject, about 0.01 mg/kg to about 10 mg/kg of body weight of the subject, for example, about 0.01 mg/kg, about 0.02 mg/kg, about 0.03 mg/kg, about 0.04 mg/kg, about 0.05 mg/kg, about 0.06 mg/kg, about 0.07 mg/kg, about 0.08 mg/kg, about 0.09 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, 1.9 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg body weight, about 20 mg/kg body weight, about 30 mg/kg body weight, about 40 mg/kg body weight, about 50 mg/kg body weight, about 60 mg/kg body weight, about 70 mg/kg body weight, about 80 mg/kg body weight, about 90 mg/kg body weight, or about 100 mg/kg body weight, inclusive of all values and ranges therebetween. In other embodiments, a suitable dosage of the AP-based agent is in a range of about 0.01 mg/kg to about 10 mg/kg of body weight, in a range of about 0.01 mg/kg to about 9 mg/kg of body weight, in a range of about 0.01 mg/kg to about 8 mg/kg of body weight, in a range of about 0.01 mg/kg to about 7 mg/kg of body weight, in a range of 0.01 mg/kg to about 6 mg/kg of body weight, in a range of about 0.05 mg/kg to about 5 mg/kg of body weight, in a range of about 0.05 mg/kg to about 4 mg/kg of body weight, in a range of about 0.05 mg/kg to about 3 mg/kg of body weight, in a range of about 0.05 mg/kg to about 2 mg/kg of body weight, in a range of about 0.05 mg/kg to about 1.5 mg/kg of body weight, or in a range of about 0.05 mg/kg to about 1 mg/kg of body weight.

In accordance with certain embodiments of the invention, the AP-based agent may be administered, for example, more than once daily (e.g., about two, about three, about four, about five, about six, about seven, about eight, about nine, or about ten times per day), about once per day, about every other day, about every third day, about once a week, about once every two weeks, about once every month, about once every two months, about once every three months, about once every six months, or about once every year.

### Methods of Treatment, Prevention, or Reduction of Side Effects of a Chemotherapuetic Treatment

In some aspects, a method of treating or preventing at least one side effect of a chemotherapeutic treatment with a nucleoside analog in a subject is provided that comprises administering to the subject an IAP, wherein the subject has undergone or is undergoing a treatment of a cancer using the chemotherapeutic treatment. The IAP is administered, simultaneously, intermittently, or in any combination thereof, with respect to the administration of the chemotherapeutic treatment with the nucleoside analog.

In various embodiments, the invention provides a method of treating, preventing, mitigating, or eliminating a side effect of a chemotherapeutic treatment in a cancer patient, comprising administering IAP to the patient. In a non-limiting example, the IAP comprises an amino acid sequence having at least about 90% sequence identity with SEQ ID NO: 41. In a further non-limiting example, the chemotherapeutic treatment comprises a nucleoside analog, optionally 5-FU. In another non-limiting example, the side effect of the chemotherapeutic treatment is diarrhea.

In various embodiments, a method of treating or preventing at least one side effect of a chemotherapeutic treatment with a nucleoside analog in a subject is provided that comprises administering to the subject an IAP, wherein the subject has undergone or is undergoing a treatment of a cancer using the chemotherapeutic treatment. In some embodiments, the effect of administering the IAP is compared to the side effect profile of a subject receiving a chemotherapeutic treatment but not receiving IAP. In a non-limiting example, the IAP comprises an amino acid sequence having at least about 90% sequence identity with SEQ ID NO: 41. In a further non-limiting example, the chemotherapeutic treatment comprises a nucleoside analog, optionally 5-FU. In another non-limiting example, the side effect of the chemotherapeutic treatment is diarrhea.

In some embodiments, the IAP is administered sequentially with the administration of the nucleoside analog, which can be prior to the administration of the nucleoside analog or after the administration of the nucleoside analog. In some cases, the administration of the IAP can at least partially overlap with the administration of the nucleoside analog. In some embodiments, the IAP is administered simultaneously with the administration of the nucleoside analog. For instance, in some embodiments, the IAP administration can begin prior to the nucleoside analog treatment (e.g., 1, 2, 3, 4, 5, 6, 7, 8, 9, 10, or more than 10 days prior to the beginning of the administration of the nucleoside analog) and can continue throughout entire nucleoside analog treatment. In some embodiments, the administration of the IAP (which can begin before or after the nucleoside analog administration begins) can continue after the completion of the administration of the nucleoside analog. For example, the administration of the IAP can continue for 1, 2, 3, 4, 5, 6, 7, 8, 9, 10, or more than 10 days after the completion of the administration of the nucleoside analog to the subject. In other embodiments, the IAP can be administered intermittently with the administration of the nucleoside analog.

In some cases, side effects, including gastrointestinal (GI) side effects of chemotherapeutic agents such as, for example, 5-FU, can be severe and life-threatening. GI side effects include, e.g., diarrhea, anorexia, nausea, emesis, stomatitis and esophagopharyngitis, and various other side effects. For example, mucositis and diarrhea are common GI side effects that are dose limiting. Various side effects associated with cancer treatments that make use of nucleoside analogs may also limit the frequency and duration of chemotherapy, thereby compromising the success of the cancer treatment.

Accordingly, the methods in accordance with some embodiments of the present disclosure can prevent, reduce, or eliminate one or more side effects associated with a chemotherapeutic treatment, including GI side effects. Non-limiting examples of side effects of the chemotherapeutic treatment comprise diarrhea, colitis, mucositis, weight loss, pain, nausea, vomiting, constipation, anemia, malnutrition, alopecia, myelosuppression, renal toxicity, hair loss, numbness, changes in tastes, loss of appetite, thinned or brittle hair, mouth sores, memory loss, hemorrhage, cardiotoxicity, hepatotoxicity, ototoxicity, and post-chemotherapy cognitive impairment.

The method in accordance with embodiments of the present disclosure, which include administration of an effective dose of an IAP, allows reducing a likelihood of onset of the side effects and reducing their length and/or severity. Also, a speed of recovery of a patient from the symptoms of the side effects can be increased.

The IAP, which can be administered orally, can be selected from human IAP or calf/bovine IAP. In some embodiments, the IAP comprises an amino acid sequence having at least about 60%, or at least about 65%, or at least about 70%, or at least about 75%, or at least about 80%, or at least about 85%, or at least about 90%, or at least about 95%, or at least about 96%, or at least about 97%, or at least about 98%, or at least about 99%, or at least about 100% identity with any one of SEQ ID NOs: 11-11, 17, 18, or 41. In some embodiments, the IAP comprises an amino acid sequence having at least about 60%, or at least about 65%, or at least about 70%, or at least about 75%, or at least about 80%, or at least about 85%, or at least about 90%, or at least about 95%, or at least about 96%, or at least about 97%, or at least about 98%, or at least about 99%, or at least about 100% identity with SEQ ID NO: 2. In some embodiments, the IAP comprises an amino acid sequence having at least 90% identity with SEQ ID NO: 2 or SEQ ID NO: 5 or SEQ ID NO: 41.

For example, in some embodiments, the methods in accordance with some embodiments of the present disclosure can prevent, reduce, or eliminate the side of the chemotherapeutic treatment, comprising diarrhea. In some embodiments, the Bristol stool scale is used to determine whether the stool from a subject administered with IAP is trending back to normal (e.g., a score of 3 or 4) from diarrhea (e.g., a score of 6 or 7). In various embodiments, the stool of a subject undergoing a chemotherapeutic treatment is scored as 6 or 7, according to the Bristol stool scale. In some embodiments, a subject undergoing a chemotherapeutic treatment and having been administered IAP has stool that is scored as 3 or 4 or 5, according to the Bristol stool scale.

The nucleoside analog can be any suitable nucleoside analog, including, without limitation, to 5-fluorouracil (5-FU), 5′-deoxyfluorouridine, 5-fluoro-2′-deoxyuridine triphosphate, fluorouridine, 2′-deoxyfluorouridine, fluorocytosine, trifluoro-methyl-2′-deoxyuridine, cytarabine, cyclocytidine, arabinosyl cytosine, 5-aza-2′-deoxycytidine, arabinosyl-5-azacytosine, 6-azacytidine, N-phosphonoacetyl-L-asparticacid, pyrazofurin, 6-azauridine, azaribine, thymidine, Fazarabine, and 3-deazauridine. In some embodiments, the nucleoside analog comprises at least one of fluoropyrimidine, 5-fluorouracil, 5-fluoro-2′-deoxycytidine, 5-fluoro-2′-deoxyuridine triphosphate, gemcitabine, troxacitabine, decitabine, cytarabine, pseudoisocytidine, Azacytidine, Decitabine, Zebularine, Ancitabine, Fazarabine, 6-azacytidine, capecitabine, N4-octadecyl-cytarabine, elaidic acid-cytarabine, cladribine, acyclovir, clofarabine, nelarabine, forodesine, 8-chloroadenosine, sapacitabine, thiarabine, and derivatives thereof. In at least some embodiments, the nucleoside analog is a pyrimidine analog such as 5-FU.

In some embodiments, the nucleoside analog is 5-fluorouracil (5-FU). In some embodiments, the standard 5-FU treatment regimen comprises administering about 12 mg/kg once daily for four successive days and optionally about 6 mg/kg on the sixth, eighth, tenth and twelfth days unless toxicity occurs. In various embodiments, administration of an IAP may allow a patient to be administered a full regimen without having to cease treatment because of side effects. In some embodiments, administration of an IAP allows a patient to be administered increased dosages (e.g. greater than about 20 mg/kg once daily, or about 18 mg/kg once daily, or about 16 mg/kg once daily, or about 14 mg/kg once daily, or about 12 mg/kg once daily) on greater than four (e.g. about 10, about 9, about 8, about 7, about 6, or about 5) successive days; greater than about 15 mg/kg once daily, or about 12 mg/kg once daily, or about 10 mg/kg once daily, or about 8 mg/kg once daily, or about 6 mg/kg once daily on the sixth, eighth, tenth and twelfth day; and/or length of administration (e.g. beyond 12 days, including, for example, about 13, about 14, about 15, about 16, about 17, about 18, about 19, about 20, about 21, about 22, about 23, about 24, about 4 weeks, or about one month).

In some embodiments, administration of an IAP may allow a patient to be administered a dose of 5-FU that exceeds about 200 mg daily, about 300 mg daily, about 400 mg daily, about 500 mg daily, about 600 mg daily, about 700 mg daily, or about 800 mg daily, or about 900 mg daily, or about 1000 mg daily.

The method of treating or preventing at least one side effect of a chemotherapeutic treatment with a nucleoside analog in a subject can be used in treatments of various cancers, including colorectal, breast, bowel, stomach, colon, anal, head and neck cancers, skin, oesophageal (gullet), and pancreatic cancer. The nucleoside analog can be (co) administered in combination of one or more of other nucleoside analogs or other therapeutic agents. For example, in some embodiments, the method further comprise administering one or more corticosteroids.

In some embodiments, the administration of the IAP allows reducing a dose, length, and/or frequency of administration of the nucleoside analog. In some embodiments, the reduced dose of the nucleoside analog can be a sub-therapeutic dose of the nucleoside analog. In some embodiments, the use of the IP allows increasing a therapeutic window of the nucleoside analog. The increased therapeutic window of the nucleoside analog may comprise one or more of increasing a subject's likelihood of receiving nucleoside analog maintenance therapy; increasing a subject's likelihood of receiving a complete regimen of the nucleoside analog; increasing a subject's likelihood of receiving more than a complete regimen of the nucleoside analog; and increasing a dose, length, and/or frequency of a nucleoside analog treatment.

In some embodiments, administering an effective amount of an IAP increases the ability of a cancer patient to receive a complete regimen of a combination therapy with 5-FU and not have to cease treatment because of, for example, side effects which are discussed in more detail below. In some embodiments, administering an effective amount of an IAP increases the ability of a cancer patient to receive a greater dose or longer duration of combination therapy with 5-FU. For example, a combination therapy regimen 5-fluorouracil/leucovorin may be (a) 6 week cycle with infusional 5-fluorouracil/leucovorin: 180 mg/m² IV infusion over 30-90 minutes once on days 1, 15, and 29 IV (infuse over 30-90 min), followed by infusion with leucovorin and 5-fluorouracil; next cycle begins on day 43 or (b) 6 week cycle with bolus 5-fluorouracil/leucovorin: 125 mg/m² on days 1, 8, 15, and 22 (infuse over 90 min), followed by bolus doses of leucovorin and 5-fluorouracil. In various embodiments, administering an effective amount of an IAP increases the dose and/or frequency of this regimen. For example, this regimen can be extended beyond 6 weeks to about 45, or about 50, or about 55, or about 60, or about 65, or about 70, or about 75, or about 100 days (including about 7, or about 8,or about 9, or about 10, or about 11, or about 12 weeks).

In some embodiments, administering an effective amount of an IAP increases the ability of a cancer patient to receive a complete regimen of any one of the FOLFOX, FOLFIRI, IFL, FL (Mayo), QUASAR, Machover schedule, CAF, CMF, ECF, and FEC regimens and not have to cease treatment because of side effects. In some embodiments, administering an effective amount of an IAP increases the ability of a cancer patient to receive a greater dose or longer duration of therapy in the FOLFOX, FOLFIRI, IFL, FL (Mayo), QUASAR, Machover schedule, CAF, CMF, ECF, and FEC regimens. In some embodiments, the present methods pertain to prevention or reduction of reduced diversity in the gut microbiome, which may be a side effect or result of chemotherapuetic treatment. In some embodiments, the present methods relate to repairing and/or repopulating the gut microbiome of a subject after chemotherapuetic treatment.

In various embodiments, the present methods prevent, reduce or eliminate a side effect of chemotherapuetic treatment, including acute side effects, long-term side effects), or cumulative side effects. In various embodiments, the present methods reduce or eliminate a local or systemic side effect of chemotherapuetic treatment. In various embodiments, the side effect of chemotherapuetic treatment is one or more of diarrhea, colitis, mucositis, weight loss, pain, nausea, vomiting, constipation, anemia, malnutrition, alopecia, myelosuppression, renal toxicity, hair loss, numbness, changes in tastes, loss of appetite, thinned or brittle hair, mouth sores, memory loss, hemorrhage, cardiotoxicity, hepatotoxicity, ototoxicity, and post-chemotherapy cognitive impairment. The side effects can also include fatigue, damage to the epithelial surfaces infertility, fibrosis, epilation, dryness (e.g. without limitation, dry mouth (xerostomia) and dry eyes (xerophthalmia), and dryness of the armpit and vaginal mucosa), lymphedema, heart disease, cognitive decline, and vitamin B12 malabsorption.

In some embodiments, an IAP is administered in combination with an additional agent, which can be any of the agents described herein or another agent, including but not limited to a corticosteroid, an antioxidant (e.g., amifostine and vitamin E), a cytokine (e.g., a stem cell factor), etc.

In various embodiments, administration of an AP-based agent stimulates and protects stem cells. For example, the present invention and composition may stimulate and protect hematopoietic stem cells including various hematopoietic progenitor cells. In another example, the present methods and compositions may stimulate and protect gastrointestinal stem cells such epithelial cells (e.g., Goblet cells) and intestinal crypt stem cells. In some embodiments, the present methods and compositions protect small intestinal cells such as Goblet cells from hypoplasia and loss (e.g., apoptosis or necrosis). In various embodiments, methods and compositions of the present invention significantly enhance recovery of the GI system following chemotherapuetic treatment.

In various embodiments, the subject is a cancer patient. The cancer can comprise colon and rectum cancer; basal cell carcinoma, biliary tract cancer; bladder cancer; bone cancer; brain and central nervous system cancer; breast cancer; cancer of the peritoneum; cervical cancer; choriocarcinoma; connective tissue cancer; cancer of the digestive system; endometrial cancer; esophageal cancer; eye cancer; cancer of the head and neck; gastric cancer (including GI cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (such as that associated with brain tumors), or Meigs' syndrome.

### Additional Therapeutic Agents and Combination Therapy

Administration of the present compositions and formulations comprising the AP-based agent may be combined with additional agents. Co-administration of the additional agent and the present compositions/formulations may be simultaneous, sequential, or combinations thereof. Further, the present compositions/formulations may comprise an additional agent (e.g., via co-formulation). For example, the additional agent and the AP-based agent may be combined into a single formulation. Alternatively, the additional agent and the AP-based agent may be formulated separately.

In one embodiment, the additional agent and the AP-based agent are administered to a subject simultaneously. The term “simultaneously” as used herein, means that the additional gent and the AP-based agent are administered with a time separation of no more than about 60 minutes, such as no more than about 30 minutes, no more than about 20 minutes, no more than about 10 minutes, no more than about 5 minutes, or no more than about 1 minute. Administration of the additional agent and the AP-based agent can be by simultaneous administration of a single formulation (e.g., a formulation comprising the additional agent and the alkaline phosphatase) or of separate formulations (e.g., a first formulation including the additional agent and a second formulation including the AP-based agent).

In a further embodiment, the additional agent and the AP-based agent are administered to a subject simultaneously but the release of the additional agent and the alkaline phosphatase from their respective dosage forms (or single unit dosage form if co-formulated) may occur sequentially.

Co-administration does not require the additional agent and the AP-based agent to be administered simultaneously, if the timing of their administration is such that the pharmacological activities of the additional agent and the AP-based agent overlap in time. For example, the additional agent and the AP-based agent can be administered sequentially. The term “sequentially” as used herein means that the additional agent and the AP-based agent are administered with a time separation of more than about 60 minutes. For example, the time between the sequential administration of the additional agent and the AP-based agent can be more than about 60 minutes, more than about 2 hours, more than about 5 hours, more than about 10 hours, more than about 1 day, more than about 2 days, more than about 3 days, or more than about 1 week apart. The administration times will depend on the rates of metabolism, excretion, and/or the pharmacodynamic activity of the additional agent and the AP-based agent being administered. Either the additional agent or the AP-based agent may be administered first.

Co-administration also does not require the additional agent and the AP-based agent to be administered to the subject by the same route of administration. Rather, each therapeutic agent can be administered by any appropriate route, for example, parenterally or non-parenterally.

In various embodiments, the additional agents include one or more of blood products, colony stimulating factors, cytokines and/or growth factors, antibiotics, diluting and/or blocking agents, mobilizing or chelating agents, stem cell transplants, and antioxidants or free radicals. Also, in embodiments in which a chemotherapy is used in combination with a radiation therapy, the additional agents include various radioprotectants.

In some embodiments, the blood product is one or more of hematopoietic growth factors, such as filgrastim (e.g. NEUPOGEN), a granulocyte colony-stimulating factor (G-CSF), which may be optionally pegylated (e.g. NEULASTA); sargramostim (LEUKINE); and a granulocyte-macrophage colony-stimulating factor (GM-CSF) and a KSF.

In some embodiments, the additional agent is one or more cytokines and/or growth factors that may confer cell protection by replenishing and/or protecting the stem cell populations. In some cases, stem cell factor (SCF, c-kit ligand), Flt-3 ligand, and/or interleukin-1 fragment IL-1b-rd may be used. Several factors, while not cytokines by nature, stimulate the proliferation of the immunocytes and may be used in combination with the AP-based agent. For example, 5-AED (5-androstenediol) is a steroid that stimulates the expression of cytokines and increases resistance to bacterial and viral infections. Synthetic compounds, such as ammonium tri-chloro (dioxoethylene-O,O′-) tellurate (AS-101), may also be used to induce secretion of numerous cytokines and for combination with the AP-based agent. Growth factors and cytokines may also be used to provide protection against the gastrointestinal syndrome. Keratinocyte growth factor (KGF) promotes proliferation and differentiation in the intestinal mucosa, and increases the post-irradiation cell survival in the intestinal crypts. Hematopoietic cytokine SCF may also increase intestinal stem cell survival and associated short-term organism survival.

In certain embodiments, the AP-based agent may be added to a regimen of cytokines (e.g. for FILGRASTIM (G-CSF) 2.5-5 μg/kg/d QD s.c. (100-200 μg/m²/d); for SARGRAMOSTIM (GM-CSF) 5-10 μg/kg/d QD s.c. (200-400 μg/m²/d); and/or for PEGFILGRASTIM (pegG-CSF) 6 mg once s.c.).

In some embodiments, the additional agent is an interleukin, such as IL-12 (e.g., HEMAMAX (NEUMEDICINES, INC.)).

In some embodiments, the antibiotic is one or more of an anti-bacterial (anti-gram positive and anti-gram negative agents), and/or anti-fungal, and/or anti-viral agent. By way of non-limiting example, in some embodiments, the antibiotic may be a quinolone, e.g. ciprofloxacin, levofloxacin, a third-or fourth-generation cephalosporin with pseudomonal coverage: e.g., cefepime, ceftazidime, or an aminoglycoside: e.g. gentamicin, amikacin, penicillin or amoxicillin, acyclovir, vanomycin. In various embodiments, the antibiotic targets Pseudomonas aeruginosa.

In some embodiments, the additional agent is a stem cell transplant (e.g. bone marrow transplant, PBSCT, MSCT). In some embodiments, the stem cell transplant is Remestemcel-L (Osiris) of CLT-008 (Cellerant).

In some embodiments, the additional agent is an antioxidant or free radical. Antioxidants and free radical scavengers that may be used in the practice of the invention include, but are not limited to, thiols, such as cysteine, cysteamine, glutathione and bilirubin; amifostine (WR-2721); vitamin A; vitamin C; vitamin E; and flavonoids such as Indian holy basil (Ocimum sanctum), orientin and vicenin.

In some embodiments, the additional agent may be a steroid (e.g. 5-androstenediol), an antioxidant (e.g., amifostine and vitamin E, gamma tocotrienol (a vitamin-E moiety), and genistein (a soy byproduct)), a cytokine (e.g., a stem cell factor), a growth factor (e.g., keratinocyte growth factor), ammonium trichloro (dioxoethylene-O,O′) tellurate, anti-nausea agents, anti-diarrhea agents, antiemetics ((e.g. oral prophylactic antiemetics) such as granisetron (KYTRIL), ondansetron (ZOFRAN), and 5-HT3 blockers with or without dexamethasone), analgesics, anxiolytics, sedatives, cytokine therapy, and antibiotics.

Further examples of combination agents include, but are not limited to, alkylating agents such as thiotepa and CYTOXAN cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (e.g., bullatacin and bullatacinone); a camptothecin (including the synthetic analogue topotecan); bryostatin; cally statin; CC-1065 (including its adozelesin, carzelesin and bizelesin synthetic analogues); cryptophycins (e.g., cryptophycin 1 and cryptophycin 8); dolastatin; duocarmycin (including the synthetic analogues, KW-2189 and CB 1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall (see, e.g., Agnew, Chem. Intl. Ed. Engl., 33:183-186 (1994)); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN doxorubicin (including morpholino-doxorubicin, cyanomorpholino-doxorubicin, 2-pyrrolino-doxorubicin and deoxy doxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as minoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; def of amine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (e.g., T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL paclitaxel (Bristol-Myers Squibb Oncology, Princeton, N.J.), ABRAXANE Cremophor-free, albumin-engineered nanoparticle formulation of paclitaxel (American Pharmaceutical Partners, Schaumberg, 111.), and TAXOTERE doxetaxel (Rhone-Poulenc Rorer, Antony, France); chloranbucil; GEMZAR gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; NAVELBINE. vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluoromethylornithine (DMFO); retinoids such as retinoic acid; capecitabine; combretastatin; leucovorin (LV); oxaliplatin, including the oxaliplatin treatment regimen (FOLFOX); lapatinib (Tykerb); inhibitors of PKC-α, Raf, H-Ras, EGFR (e.g., erlotinib (Tarceva) and VEGF-A that reduce cell proliferation and pharmaceutically acceptable salts, acids or derivatives of any of the above. In addition, the methods of treatment can further include the use of radiation. In addition, the methods of treatment can further include the use of photodynamic therapy.

### Definitions

As used herein, “a,” “an,” or “the” can mean one or more than one.

Further, the term “about” when used in connection with a referenced numeric indication means the referenced numeric indication plus or minus up to 10% of that referenced numeric indication. For example, the language “about 50%” covers the range of 45% to 55%.

An “effective amount,” when used in connection with medical uses is an amount that is effective for providing a measurable treatment, prevention, or reduction in the rate of pathogenesis of a disorder of interest.

As used herein, something is “decreased” if a read-out of activity and/or effect is reduced by a significant amount, such as by at least about 10%, at least about 20%, at least about 30%, at least about 40%, at least about 50%, at least about 60%, at least about 70%, at least about 80%, at least about 90%, at least about 95%, at least about 97%, at least about 98%, or more, up to and including at least about 100%, in the presence of an agent or stimulus relative to the absence of such modulation. As will be understood by one of ordinary skill in the art, in some embodiments, activity is decreased and some downstream read-outs will decrease but others can increase.

Conversely, activity is “increased” if a read-out of activity and/or effect is increased by a significant amount, for example by at least about 10%, at least about 20%, at least about 30%, at least about 40%, at least about 50%, at least about 60%, at least about 70%, at least about 80%, at least about 90%, at least about 95%, at least about 97%, at least about 98%, or more, up to and including at least about 100% or more, at least about 2-fold, at least about 3-fold, at least about 4-fold, at least about 5-fold, at least about 6-fold, at least about 7-fold, at least about 8-fold, at least about 9-fold, at least about 10-fold, at least about 50-fold, at least about 100-fold, in the presence of an agent or stimulus, relative to the absence of such agent or stimulus.

As referred to herein, all compositional percentages are by weight of the total composition, unless otherwise specified. As used herein, the word “include,” and its variants, is intended to be non-limiting, such that recitation of items in a list is not to the exclusion of other like items that may also be useful in the compositions and methods of this technology. Similarly, the terms “can” and “may” and their variants are intended to be non-limiting, such that recitation that an embodiment can or may comprise certain elements or features does not exclude other embodiments of the present technology that do not contain those elements or features.

Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

As used herein, the words “preferred” and “preferably” refer to embodiments of the technology that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the technology.

In some embodiments, the terms “patient” and “subject” are used interchangeably. In some embodiments, the subject and/or animal is a mammal, e.g., a human, mouse, rat, guinea pig, dog, cat, horse, cow, pig, rabbit, sheep, or non-human primate, such as a monkey, chimpanzee, or baboon. In other embodiments, the subject and/or animal is a non-mammal, such, for example, a zebrafish.

In various embodiments, methods of the invention are useful in treatment a human subject. In some embodiments, the human is a pediatric human. In other embodiments, the human is an adult human. In other embodiments, the human is a geriatric human. In other embodiments, the human may be referred to as a patient. In some embodiments, the human is a female. In some embodiments, the human is a male.

In certain embodiments, the human has an age in a range of from about 1 to about 18 months old, from about 18 to about 36 months old, from about 1 to about 5 years old, from about 5 to about 10 years old, from about 10 to about 15 years old, from about 15 to about 20 years old, from about 20 to about 25 years old, from about 25 to about 30 years old, from about 30 to about 35 years old, from about 35 to about 40 years old, from about 40 to about 45 years old, from about 45 to about 50 years old, from about 50 to about 55 years old, from about 55 to about 60 years old, from about 60 to about 65 years old, from about 65 to about 70 years old, from about 70 to about 75 years old, from about 75 to about 80 years old, from about 80 to about 85 years old, from about 85 to about 90 years old, from about 90 to about 95 years old or from about 95 to about 100 years old.

The amount of compositions described herein needed for achieving a therapeutic effect may be determined empirically in accordance with conventional procedures for the particular purpose. Generally, for administering therapeutic agents (e.g., microbiome-modulating agents and/or additional therapeutic agents described herein) for therapeutic purposes, the therapeutic agents are given at a pharmacologically effective dose. A “pharmacologically effective amount,” “pharmacologically effective dose,” “therapeutically effective amount,” or “effective amount” refers to an amount sufficient to produce the desired physiological effect or amount capable of achieving the desired result, particularly for treating the disorder or disease. An effective amount as used herein would include an amount sufficient to, for example, delay the development of a symptom of the disorder or disease, alter the course of a symptom of the disorder or disease (e.g., slow the progression of a symptom of the disease), reduce or eliminate one or more symptoms or manifestations of the disorder or disease, and reverse a symptom of a disorder or disease. Therapeutic benefit also includes halting or slowing the progression of the underlying disease or disorder, regardless of whether improvement is realized.

Effective amounts, toxicity, and therapeutic efficacy can be determined by standard pharmaceutical procedures in cell cultures, tissue samples, tissue homogenates or experimental animals, e.g., for determining the LD50 (the dose lethal to about 50% of the population) and the ED50 (the dose therapeutically effective in about 50% of the population). The dosage can vary depending upon the dosage form employed and the route of administration utilized. The dose ratio between toxic and therapeutic effects is the therapeutic index and can be expressed as the ratio LD50/ED50. In some embodiments, compositions and methods that exhibit large therapeutic indices are preferred. A therapeutically effective dose can be estimated initially from in vitro assays, including, for example, cell culture assays or measurements or methane production in stool samples. Also, a dose can be formulated in animal models to achieve a circulating plasma concentration range that includes the IC50 as determined in cell culture, or in an appropriate animal model. Levels of the described compositions in plasma can be measured, for example, by high performance liquid chromatography. The effects of any particular dosage can be monitored by a suitable bioassay. The dosage can be determined by a physician and adjusted, as necessary, to suit observed effects of the treatment.

In certain embodiments, the effect will result in a quantifiable change of at least about 10%, at least about 20%, at least about 30%, at least about 50%, at least about 70%, or at least about 90%. In some embodiments, the effect will result in a quantifiable change of about 10%, about 20%, about 30%, about 50%, about 70%, or even about 90% or more. Therapeutic benefit also includes halting or slowing the progression of the underlying disease or disorder, regardless of whether improvement is realized.

As used herein, “methods of treatment” are equally applicable to use of a composition for treating the diseases or disorders described herein and/or compositions for use and/or uses in the manufacture of a medicaments for treating the diseases or disorders described herein.

### EXAMPLES

### Example 1: IAP Reduces Side Effects of 5-FU

Experiments were performed in order to assess the effect of IAP treatment on diarrhea and weight change side effects of using a chemotherapeutic agent comprising a nucleoside analog for treating cancer.

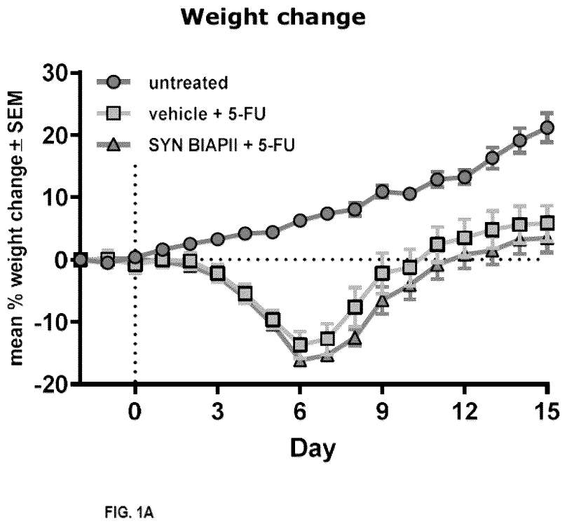

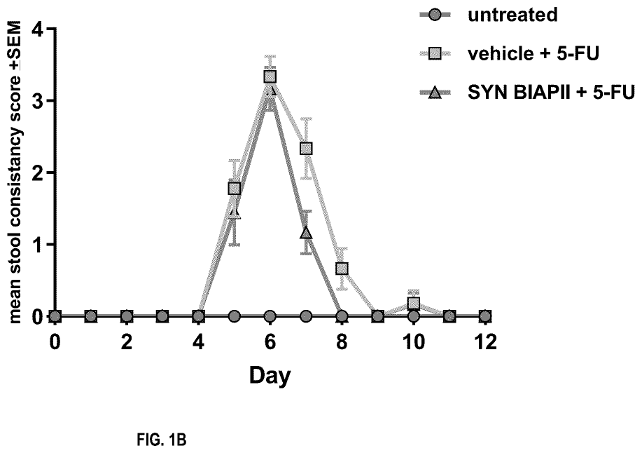

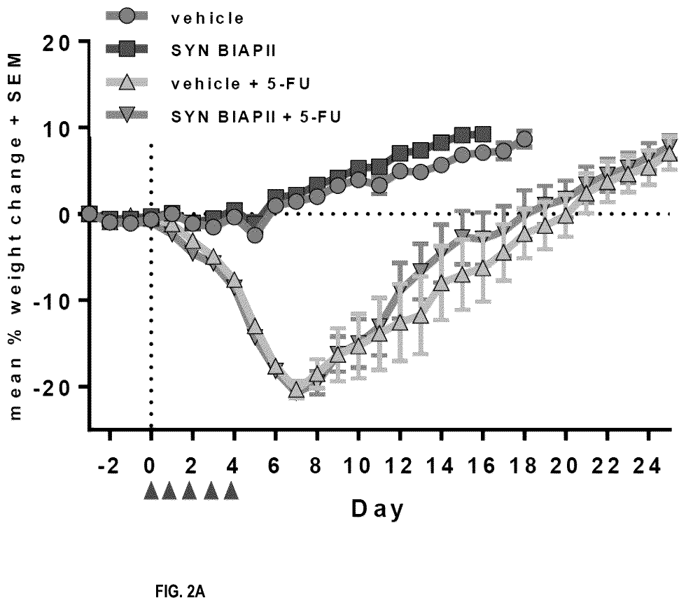

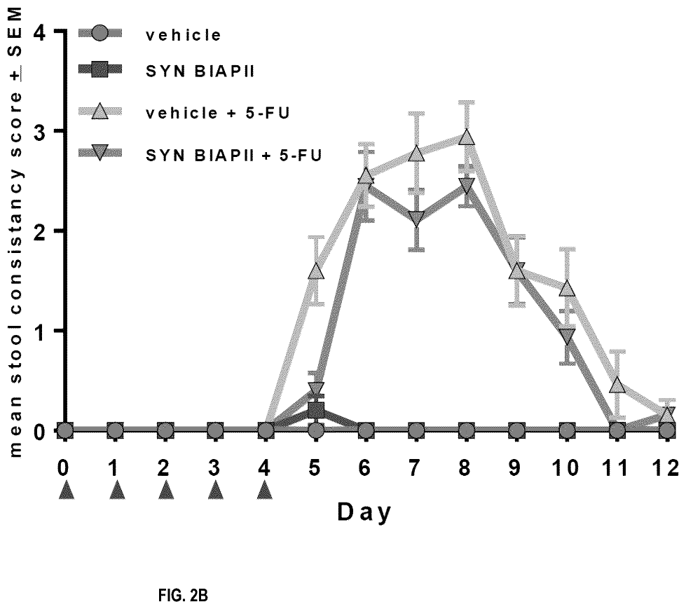

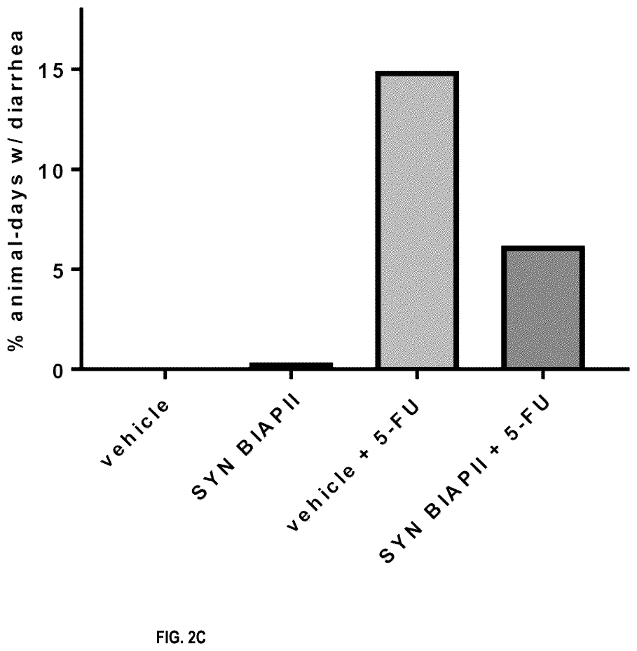

The study conducted included evaluation of the effects of administration of an IAP (“SYN-020” is synonymous with IAP and SYN BIAPII) along with a chemotherapeutic treatment with 5-fluorouracil (5-FU) in a murine model. Taken together, FIGS. 1A-1B and 2A-2C illustrate, based on assessment of mice weight change and stool consistency, that administration of IAP reduces side effects of 5-FU. Stool consistency was scored as the following: Score 0=Normal (no diarrhea); Score 2=Loose, formed stools; and Score 4=Diarrhea.

The first set of experiments included three groups of BALB/c mice inoculated with CT26 murine colorectal cancer cells into the right flank: (1) a no-treatment control group of 18 male mice (“untreated”); (2) a group of 18 male mice (“vehicle +5-FU”) that received an intraperitoneal (IP) QD (once daily) dose of 30 mg/kg of 5-FU for 5 consecutive days (days 0-4) and a vehicle was administered PO (orally) BID (twice a day) throughout the study (days −3 to +26); and (3) a group of 18 male mice (“SYN BIAPII+5-FU”) that received an IP QD dose of 30 mg/kg of 5-FU for 5 consecutive days (days 0-4) and an IAP (SYN-020) in a dose of 0.5 mg/kg, administered PO BID throughout the study (days −3 to +26).

FIG. 1A shows mean percent of weight change ±SEM for untreated (top curve), vehicle+5-FU (middle curve), and SYN BIAPII+5-FU (bottom curve) mice versus a number of days of the study. FIG. 1B shows assessment of stool consistency, as a mean stool consistency score ±SEM versus a number of days, of vehicle+5-FU and SYN BIAPII+5-FU mice relative to untreated mice data for which is shown as a baseline at the x-axis). As shown, the administration of 5-FU caused loose stools/diarrhea, as well as associated weight loss, in all of the treated animals. These symptoms peaked at day 6 in both of the vehicle+5-FU and SYN BIAPII+5-FU mice groups, and recovered by day 9. The administration of the IAP in the SYN BIAPII+5-FU group shows a significantly faster recovery from the loose stools/diarrhea symptoms and associated weight loss (p<0.03).

The second set of experiments included four groups of BALB/c mice inoculated with CT26 murine colorectal cancer cells into the right flank: (1) a vehicle control group of 30 male mice (“vehicle”); (2) a group of 30 male mice that received a daily treatment dose of 100U/dose, 0.5 mg/kg of SYN BIAP II alone (“SYN BIAP II”); (3) a group of 30 male mice (“vehicle+5-FU”) that received an IP QD dose of 30 mg/kg of 5-FU for 5 consecutive days (days 0-4) and vehicle treatment; and (4) a group of 30 male mice (“SYN BIAPII+5-FU”) that received an IP QD dose of 30 mg/kg of 5-FU for 5 consecutive days (days 0-4) and an IAP (SYN-020) in a dose of 100U/dose, 0.5 mg/kg, administered PO BID throughout the study (days −3 to +26).

FIG. 2A depicts the mean daily body weight through 50% group survival, which is displayed as percent change over time compared to body weight at Day 0+SEM. Animals administered vehicle+5-FU over Days 0-4 displayed chemotherapy-induced weight loss that peaked at about (−20%) on Day 7, after which animals regained lost weight by about Day 20. The mean percentage of weight change of animals administered SYN BIAPII+5-FU is not significantly different from that of animals administered vehicle+5-FU. Cumulative differences in body weight change between treatment groups through 50% common group survival (Day (−3)-16) were assessed by calculating the area under the curve (AUC) for each animal using the trapezoidal transformation.

FIG. 2B shows the results of Stool Consistency Scoring daily over Days 0-12; mean scores+SEM are shown. Arrows indicate 5-FU administration days. Animals treated with vehicle or SYN BIAPII alone generally displayed normal stool consistency throughout the course of the analysis. For animals administered vehicle+5-FU or SYN BIAPII+5-FU, incidence of loose stools or diarrhea was first observed on Day 5. For animals administered vehicle+5-FU, incidence of loose stools generally resolved by Day 12; for animals administered SYN BIAPII+5-FU, incidence of loose stools generally resolved by Day 11. Compared to treatment with vehicle+5-FU, an improvement in overall stool score was observed for animals treated with SYN BIAPII+5-FU over days 5-11. The statistical significance of group differences in daily stool scores was assessed on each day by Mann-Whitney rank-sum analysis. Compared to treatment with vehicle+5-FU, animals treated with SYN BIAPII+5-FU showed a statistically significant improvement in overall stool score on Day 5 (p=0.006).

Table 1 below compares the duration of 5-FU-induced diarrhea by calculating the percentage of total animal-days with diarrhea (score=4). Groups were compared by chi-square test. Compared to untreated animals, a statistically significant cumulative increase in animal-days with diarrhea was observed both for animals treated with vehicle+5-FU (p=<0.0001) and for those treated with SYN BIAPII+5-FU (p=<0.0001). Compared to animals treated with vehicle+5-FU, animals treated with SYN BIAPII+5-FU displayed a statistically significant improvement in overall duration of diarrhea (p=0.0006).

TABLE 1
Comparison of Duration of Diarrhea amoung Groups 1-4
Duration of Diarrhea (score = 4)

| | | | | Total | | Chi-Sq. | | Chi-Sq. | |
|---|---|---|---|---|---|---|---|---|---|
| | | Days = | Days < | Animal | % Days = | vs. | p value vs. | vs. | p value vs. |
| Group | Treatment | 4 | 4 | Days | 4 | Group 1; df | Group 1 | Group 3 | Group 3 |
| 1 | vehicle | 0 | 306 | 306 | 0 | — | — | — | — |
| 2 | SYN BIAPII | 1 | 299 | 300 | 0.33% | 1.022; 1 | 0.312 | — | — |
| 3 | vehicle + 5-FU | 43 | 245 | 288 | 14.93% | 49.25; 1 | <0.0001**** | — | — |
| 4 | SYN BIAPII + | 18 | 273 | 291 | 6.19% | 19.52; 1 | <0.0001**** | 11.74; 1 | 0.0006*** |
| | 5-FU | | | | | | | | |

Finally, FIG. 2C depicts a graphical representation of the percentage of total animal-days with diarrhea by treatment group.

Accordingly, the present studies demonstrate that administration of IAP improves side effects associated with 5-FU, such as, e.g., diarrhea and mucositis. Accordingly, IAP can be utilized as a therapeutic agent for use in treatment, prevention, and mitigation of cancer treatment-related side effects and to enhance the overall response of chemotherapeutic agents.

### EQUIVALENTS

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

Those skilled in the art will recognize, or be able to ascertain, using no more than routine experimentation, numerous equivalents to the specific embodiments described specifically herein. Such equivalents are intended to be encompassed in the scope of the following claims.

### INCORPORATION BY REFERENCE

All patents and publications referenced herein are hereby incorporated by reference in their entireties.

The publications discussed herein are provided solely for their disclosure prior to the filing date of the present application. Nothing herein is to be construed as an admission that the present invention is not entitled to antedate such publication by virtue of prior invention.

As used herein, all headings are simply for organization and are not intended to limit the disclosure in any manner. The content of any individual section may be equally applicable to all sections.

1. A method of treating or preventing at least one side effect of a chemotherapeutic treatment with a nucleoside analog in a subject, comprising administering to the subject an intestinal alkaline phosphatase (IAP), wherein the subject has undergone or is undergoing a treatment of a cancer using the chemotherapeutic treatment.

2. The method of claim 1, wherein the IAP is administered sequentially with the chemotherapeutic treatment.

3. The method of claim 2, wherein the IAP is administered prior to the chemotherapeutic treatment.

4. The method of claim 2, wherein the IAP is administered after the chemotherapeutic treatment.

5. The method of claim 1, wherein the IAP is administered simultaneously with the chemotherapeutic treatment.

6. The method of claim 1, wherein the IAP is administered intermittently with the chemotherapeutic treatment.

7. The method of any one of claims 1 to 6, wherein the side effect of the chemotherapeutic treatment comprises diarrhea, colitis, mucositis, weight loss, pain, nausea, vomiting, constipation, anemia, malnutrition, alopecia, myelosuppression, renal toxicity, hair loss, numbness, changes in tastes, loss of appetite, thinned or brittle hair, mouth sores, memory loss, hemorrhage, cardiotoxicity, hepatotoxicity, ototoxicity, and post-chemotherapy cognitive impairment.

8. The method of any one of claims 1 to 7, wherein the side effect of the chemotherapeutic treatment comprises a gastrointestinal (GI) side effect.

9. The method of claim 1 or 8, wherein the side effect comprises mucositis.

10. The method of any one of claims 1 to 9, wherein the side effect comprises diarrhea.

11. The method of any one of claims 1 to 10, wherein the IAP is selected from human IAP or calf/bovine IAP.

12. The method of any one of claims 1 to 11, wherein the IAP comprises an amino acid sequence having at least about 60%, or at least about 65%, or at least about 70%, or at least about 75%, or at least about 80%, or at least about 85%, or at least about 90%, or at least about 95%, or at least about 96%, or at least about 97%, or at least about 98%, or at least about 99%, or at least about 100% identity with any one of SEQ ID NOs: 1-11, 17, 18, or 41.

13. The method of any one of claims 1 to 11, wherein the IAP comprises an amino acid sequence having at least about 60%, or at least about 65%, or at least about 70%, or at least about 75%, or at least about 80%, or at least about 85%, or at least about 90%, or at least about 95%, or at least about 96%, or at least about 97%, or at least about 98%, or at least about 99%, or at least about 100% identity with SEQ ID NO: 2, SEQ ID NO: 5, or SEQ ID NO: 41.

14. The method of any one of claims 1 to 11, wherein the IAP comprises an amino acid sequence having at least 90% identity with SEQ ID NO: 2, SEQ ID NO: 5, or SEQ ID NO: 41.

15. The method of any one of claims 1 to 14, wherein the IAP is administered orally.

16. The method of any one of claims 1 to 15, wherein the IAP is formulated for gastrointestinal (GI) release.

17. The method of any one of claims 1 to 16, wherein the nucleoside analog is a purine or pyrimidine analog or derivatives thereof.

18. The method of claim 1, wherein the nucleoside analog is a pyrimidine analog.

19. The method of claim 1, wherein the pyrimidine analog comprises at least one of 5-fluorouracil, 5′-deoxyfluorouridine, 5-fluoro-2′-deoxyuridine triphosphate, fluorouridine, 2′-deoxyfluorouridine, fluorocytosine, trifluoro-methyl-2′-deoxyuridine, cytarabine, cyclocytidine, arabinosyl cytosine, 5-aza-2′-deoxycytidine, arabinosyl-5-azacytosine, 6-azacytidine, N-phosphonoacetyl-L-asparticacid, pyrazofurin, 6-azauridine, azaribine, thymidine, Fazarabine, 3-deazauridine, and derivatives thereof.

20. The method of claim 19, wherein the pyrimidine analog is 5-fluorouracil.

21. The method of any one of claims 1 to 16, wherein the nucleoside analog comprises at least one of fluoropyrimidine, 5-fluorouracil, 5-fluoro-2′-deoxycytidine, 5-fluoro-2′-deoxyuridine triphosphate, gemcitabine, troxacitabine, decitabine, cytarabine, pseudoisocytidine, Azacytidine, Decitabine, Zebularine, Ancitabine, Fazarabine, 6-azacytidine, capecitabine, N4-octadecyl-cytarabine, elaidic acid-cytarabine, cladribine, acyclovir, clofarabine, nelarabine, forodesine, 8-chloroadenosine, sapacitabine, thiarabine, and derivatives thereof.

22. The method of any one above claims 1 to 21, wherein the subject has cancer.

23. The method of claim 22, wherein the cancer comprises basal cell carcinoma; biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including GI cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (such as that associated with brain tumors), or Meigs' syndrome.

24. The method of any one of claims 1 to 23, wherein the administration of the IAP allows reducing a dose, length, and/or frequency of administration of the nucleoside analog.

25. The method of claim 24, wherein the reduced dose of the nucleoside analog is a sub-therapeutic dose of the nucleoside analog.

26. The method of any one of claims 1 to 25, wherein the method increases a therapeutic window of the nucleoside analog.

27. The method of claim 26, wherein the increased therapeutic window of the nucleoside analog comprises one or more of increasing a subject's likelihood of receiving nucleoside analog maintenance therapy; increasing a subject's likelihood of receiving a complete regimen of the nucleoside analog; increasing a subject's likelihood of receiving more than a complete regimen of the nucleoside analog; and increasing a dose, length, and/or frequency of a nucleoside analog treatment.

28. The method of any one of claims 1 to 27 wherein the IAP does not hinder the treatment of the cancer in the subject.

29. The method of claim 1, wherein
the IAP comprises an amino acid sequence having at least about the IAP comprises an amino acid sequence having at least about 60%, or at least about 65%, or at least about 70%, or at least about 75%, or at least about 80%, or at least about 85%, or at least about 90%, or at least about 95%, or at least about 96%, or at least about 97%, or at least about 98%, or at least about 99%, or at least about 100% identity with SEQ ID NO: 41;
the chemotherapeutic treatment is 5-FU; and.
the side effect of the chemotherapeutic treatment comprises a gastrointestinal (GI) side effect.

30. The method of claim 29, wherein the gastrointestinal (GI) side effect is diarrhea.

31. The method of claim 29, wherein the gastrointestinal (GI) side effect is mucositis.

32. The method of any one of claim 1-31, wherein
the IAP comprises an amino acid sequence having at least about the IAP comprises an amino acid sequence having at least about 60%, or at least about 65%, or at least about 70%, or at least about 75%, or at least about 80%, or at least about 85%, or at least about 90%, or at least about 95%, or at least about 96%, or at least about 97%, or at least about 98%, or at least about 99%, or at least about 100% identity with SEQ ID NO: 41;
the chemotherapeutic treatment is 5-FU; and.
the side effect of the chemotherapeutic treatment comprises a gastrointestinal (GI) side effect.

33. The method of claim 32, wherein the gastrointestinal (GI) side effect is diarrhea.

34. The method of claim 32, wherein the gastrointestinal (GI) side effect is mucositis.


SEQUENCE LISTING (41 sequences)

SEQ ID NO: 1 - protein, 528 aa, synthetic construct
  REGION 1..528: note = Synthetic polymer.
MQGPWVLLLLGLRLQLSLGVIPAEEENPAFWNRQAAEALDAAKKLQPIQKVAKNLILFLGDGLGVPTVTATRILKGQKNGKLGPETPLAMDRFPYLALSKTYNVDRQVPDSAATATAYLCGVKANFQTIGLSAAARFNQCNTTRGNEVISVMNRAKQAGKSVGVVTTTRVQHASPAGTYAHTVNRNWYSDADMPASARQEGCQDIATQLISNMDIDVILGGGRKYMFPMGTPDPEYPADASQNGIRLDGKNLVQEWLAKHQGAWYVWNRTELMQASLDQSVTHLMGLFEPGDTKYEIHRDPTLDPSLMEMTEAALRLLSRNPRGFYLFVEGGRIDHGHHEGVAYQALTEAVMFDDAIERAGQLTSEEDTLTLVTADHSHVFSFGGYTLRGSSIFGLAPSKAQDSKAYTSILYGNGPGYVFNSGVRPDVNESESGSPDYQQQAAVPLSSETHGGEDVAVFARGPQAHLVHGVQEQSFVAHVMAFAACLEPYTACDLAPPACTTDAAHPVAASLPLLAGTLLLLGASAAP

SEQ ID NO: 2 - protein, 420 aa, synthetic construct
  REGION 1..420: note = Synthetic polymer.
MQGACVLLLLGLHLQLSLGLIPAEEENPAFWNRQAAQALDVAKKLQPIQTAAKNVILFLGDGMGVPTVTATRILKGQMNGKLGPETPLAMDQFPYVALSKTYNVDRQVPDSAGTATAYLCGVKGNYRTIGVSAAARYNQCNTTRGNEVTSVINRAKKAGKAVGVVTTTRVQHASPAGAYAHTVNRNWYSDADLPADAQKNGCQDIAAQLVYNMDIDVILGGGRMYMFPEGTPDPEYPDDASVNGVRKDKQNLVQEWQAKHQGAQYVWNRTALLQAADDSSVTHLMGLFEPADMKYNVQQDHTKDPTLAEMTEAALQVLSRNPRGFYLFVEGGRIDHGHHDGKAYMALTEAIMFDNAIAKANELTSELDTLILVTADHSHVFSFGGYTLRGTSIFGLAPGKALDSKSYTSILYGNGPGYAL

SEQ ID NO: 3 - protein, 530 aa, synthetic construct
  REGION 1..530: note = Synthetic polymer.
MQWACVLLLLGLWLQLSLTFIPAEEEDPAFWNRQAAQALDVAKKLQPIQTAAKNVILFLGDGMGVPTVTATRILKGQMNGKLGPETPLAMDQFPYVALSKTYNVDRQVPDSAGTATAYLCGVKGNYKTIGVSAAARYNQCNTTSGNEVTSVMNRAKKAGKSVGVVTTSRVQHASPAGAYAHTVNRNWYSDADLPADAQTYGCQDIATQLVNNMDIDVILGGGRMYMFPEGTPDPEYPYDVNQTGVRKDKRNLVQEWQAKHQGAQYVWNRTELLQAANDPSVTHLMGLFEPADMKYNVQQDPTKDPTLEEMTEAALQVLSRNPQGFYLFVEGGRIDHGHHEGKAYMALTDTVMFDNAIAKANELTSELDTLILATADHSHVFSFGGYTLRGTSIFGLAPSKASDNKSYTSILYGNGPGYVLGGGLRPDVNDSISEDPSYRQQAAVPLSSESHGGEDVAVFARGPQAHLVHGVQEETFVAHVMAFAGCVEPYTDCNLPAPSGLSDAAHLAASPPSLALLAGAMLLLLAPALY

SEQ ID NO: 4 - protein, 503 aa, synthetic construct
  REGION 1..503: note = Synthetic polymer.
MQGPWVLLLLGLRLQLSLGVIPAEEENPAFWNRQAAEALDAAKKLQPIQKVAKNLILFLGDGLGVPTVTATRILKGQKNGKLGPETPLAMDRFPYLALSKTYNVDRQVPDSAATATAYLCGVKANFQTIGLSAAARFNQCNTTRGNEVISVMNRAKQAGKSVGVVTTTRVQHASPAGTYAHTVNRNWYSDADMPASARQEGCQDIATQLISNMDIDVILGGGRKYMFPMGTPDPEYPADASQNGIRLDGKNLVQEWLAKHQGAWYVWNRTELMQASLDQSVTHLMGLFEPGDTKYEIHRDPTLDPSLMEMTEAALRLLSRNPRGFYLFVEGGRIDHGHHEGVAYQALTEAVMFDDAIERAGQLTSEEDTLTLVTADHSHVFSFGGYTLRGSSIFGLAPSKAQDSKAYTSILYGNGPGYVFNSGVRPDVNESESGSPDYQQQAAVPLSSETHGGEDVAVFARGPQAHLVHGVQEQSFVAHVMAFAACLEPYTACDLAPPAGTTD

SEQ ID NO: 5 - protein, 506 aa, synthetic construct
  REGION 1..506: note = Synthetic polymer.
MQGACVLLLLGLHLQLSLGLIPAEEENPAFWNRQAAQALDVAKKLQPIQTAAKNVILFLGDGMGVPTVTATRILKGQMNGKLGPETPLAMDQFPYVALSKTYNVDRQVPDSAGTATAYLCGVKGNYRTIGVSAAARYNQCNTTRGNEVTSVINRAKKAGKAVGVVTTTRVQHASPAGAYAHTVNRNWYSDADLPADAQKNGCQDIAAQLVYNMDIDVILGGGRMYMFPEGTPDPEYPDDASVNGVRKDKQNLVQEWQAKHQGAQYVWNRTALLQAADDSSVTHLMGLFEPADMKYNVQQDHTKDPTLAEMTEAALQVLSRNPRGFYLFVEGGRIDHGHHDGKAYMALTEAIMFDNAIAKANELTSELDTLILVTADHSHVFSFGGYTLRGTSIFGLAPGKALDSKSYTSILYGNGPGYALGGGSRPDVNGSTSEEPSYRQQAAVPLASETHGGEDVAVFARGPQAHLVHGVQEETFVAHIMAFAGCVEPYTDCNLPAPATATSIPD

SEQ ID NO: 6 - protein, 503 aa, synthetic construct
  REGION 1..503: note = Synthetic polymer.
MQWACVLLLLGLWLQLSLTFIPAEEEDPAFWNRQAAQALDVAKKLQPIQTAAKNVILFLGDGMGVPTVTATRILKGQMNGKLGPETPLAMDQFPYVALSKTYNVDRQVPDSAGTATAYLCGVKGNYKTIGVSAAARYNQCNTTSGNEVTSVMNRAKKAGKSVGVVTTSRVQHASPAGAYAHTVNRNWYSDADLPADAQTYGCQDIATQLVNNMDIDVILGGGRMYMFPEGTPDPEYPYDVNQTGVRKDKRNLVQEWQAKHQGAQYVWNRTELLQAANDPSVTHLMGLFEPADMKYNVQQDPTKDPTLEEMTEAALQVLSRNPQGFYLFVEGGRIDHGHHEGKAYMALTDTVMFDNAIAKANELTSELDTLILATADHSHVFSFGGYTLRGTSIFGLAPSKASDNKSYTSILYGNGPGYVLGGGLRPDVNDSISEDPSYRQQAAVPLSSESHGGEDVAVFARGPQAHLVHGVQEETFVAHVMAFAGCVEPYTDCNLPAPSGLSD

SEQ ID NO: 7 - protein, 508 aa, synthetic construct
  REGION 1..508: note = Synthetic polymer.
MQWACVLLLLGLWLQLSLTFIPAEEEDPAFWNRQAAQALDVAKKLQPIQTAAKNVILFLGDGMGVPTVTATRILKGQMNGKLGPETPLAMDQFPYVALSKTYNVDRQVPDSAGTATAYLCGVKGNYKTIGVSAAARYNQCNTTSGNEVTSVMNRAKKAGKSVGVVTTSRVQHASPAGAYAHTVNRNWYSDADLPADAQTYGCQDIATQLVNNMDIDVILGGGRMYMFPEGTPDPEYPYDVNQTGVRKDKRNLVQEWQAKHQGAQYVWNRTELLQAANDPSVTHLMGLFEPADMKYNVQQDPTKDPTLEEMTEAALQVLSRNPQGFYLFVEGGRIDHGHHEGKAYMALTDTVMFDNAIAKANELTSELDTLILATADHSHVFSFGGYTLRGTSIFGLAPSKASDNKSYTSILYGNGPGYVLGGGLRPDVNDSISEDPSYRQQAAVPLSSESHGGEDVAVFARGPQAHLVHGVQEETFVAHVMAFAGCVEPYTDCNLPAPSGLSDAAHLA

SEQ ID NO: 8 - protein, 754 aa, synthetic construct
  REGION 1..754: note = Synthetic polymer.
MQGACVLLLLGLHLQLSLGLIPAEEENPAFWNRQAAQALDVAKKLQPIQTAAKNVILFLGDGMGVPTVTATRILKGQMNGKLGPETPLAMDQFPYVALSKTYNVDRQVPDSAGTATAYLCGVKGNYRTIGVSAAARYNQCNTTRGNEVTSVINRAKKAGKAVGVVTTTRVQHASPAGAYAHTVNRNWYSDADLPADAQKNGCQDIAAQLVYNMDIDVILGGGRMYMFPEGTPDPEYPDDASVNGVRKDKQNLVQEWQAKHQGAQYVWNRTALLQAADDSSVTHLMGLFEPADMKYNVQQDHTKDPTLAEMTEAALQVLSRNPRGFYLFVEGGRIDHGHHDGKAYMALTEAIMFDNAIAKANELTSELDTLILVTADHSHVFSFGGYTLRGTSIFGLAPGKALDSKSYTSILYGNGPGYALGGGSRPDVNGSTSEEPSYRQQAAVPLASETHGGEDVAVFARGPQAHLVHGVQEETFVAHIMAFAGCVEPYTDCNLPAPATATSIPDGGSGGSGGGGSGGGGSEPKSCDKTHTCPPCPAPELLGGPSVFLFPPKPKDTLMISRTPEVTCVVVDVSHEDPQVKFNWYVDGVQVHNAKTKPREQQYNSTYRVVSVLTVLHQNWLDGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 751 aa, synthetic construct
  REGION 1..751: note = Synthetic polymer.
MQWACVLLLLGLWLQLSLTFIPAEEEDPAFWNRQAAQALDVAKKLQPIQTAAKNVILFLGDGMGVPTVTATRILKGQMNGKLGPETPLAMDQFPYVALSKTYNVDRQVPDSAGTATAYLCGVKGNYKTIGVSAAARYNQCNTTSGNEVTSVMNRAKKAGKSVGVVTTSRVQHASPAGAYAHTVNRNWYSDADLPADAQTYGCQDIATQLVNNMDIDVILGGGRMYMFPEGTPDPEYPYDVNQTGVRKDKRNLVQEWQAKHQGAQYVWNRTELLQAANDPSVTHLMGLFEPADMKYNVQQDPTKDPTLEEMTEAALQVLSRNPQGFYLFVEGGRIDHGHHEGKAYMALTDTVMFDNAIAKANELTSELDTLILATADHSHVFSFGGYTLRGTSIFGLAPSKASDNKSYTSILYGNGPGYVLGGGLRPDVNDSISEDPSYRQQAAVPLSSESHGGEDVAVFARGPQAHLVHGVQEETFVAHVMAFAGCVEPYTDCNLPAPSGLSDGGSGGSGGGGSGGGGSEPKSCDKTHTCPPCPAPELLGGPSVFLFPPKPKDTLMISRTPEVTCVVVDVSHEDPQVKFNWYVDGVQVHNAKTKPREQQYNSTYRVVSVLTVLHQNWLDGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 539 aa, synthetic construct
  REGION 1..539: note = Synthetic polymer.
MQWACVLLLLGLWLQLSLTFIPAEEEDPAFWNRQAAQALDVAKKLQPIQTAAKNVILFLGDGMGVPTVTATRILKGQMNGKLGPETPLAMDQFPYVALSKTYNVDRQVPDSAGTATAYLCGVKGNYKTIGVSAAARYNQCNTTSGNEVTSVMNRAKKAGKSVGVVTTSRVQHASPAGAYAHTVNRNWYSDADLPADAQTYGCQDIATQLVNNMDIDVILGGGRMYMFPEGTPDPEYPYDVNQTGVRKDKRNLVQEWQAKHQGAQYVWNRTELLQAANDPSVTHLMGLFEPADMKYNVQQDPTKDPTLEEMTEAALQVLSRNPQGFYLFVEGGRIDHGHHEGKAYMALTDTVMFDNAIAKANELTSELDTLILATADHSHVFSFGGYTLRGTSIFGLAPSKASDNKSYTSILYGNGPGYVLGGGLRPDVNDSISEDPSYRQQAAVPLSSESHGGEDVAVFARGPQAHLVHGVQEETFVAHVMAFAGCVEPYTDCNLEVLFQGPAPPAGTTDAAHPGRSVVPALLPLRAGTLLLLETATAP

SEQ ID NO: 11 - protein, 534 aa, synthetic construct
  REGION 1..534: note = Synthetic polymer.
MQWACVLLLLGLWLQLSLTFIPAEEEDPAFWNRQAAQALDVAKKLQPIQTAAKNVILFLGDGMGVPTVTATRILKGQMNGKLGPETPLAMDQFPYVALSKTYNVDRQVPDSAGTATAYLCGVKGNYKTIGVSAAARYNQCNTTSGNEVTSVMNRAKKAGKSVGVVTTSRVQHASPAGAYAHTVNRNWYSDADLPADAQTYGCQDIATQLVNNMDIDVILGGGRMYMFPEGTPDPEYPYDVNQTGVRKDKRNLVQEWQAKHQGAQYVWNRTELLQAANDPSVTHLMGLFEPADMKYNVQQDPTKDPTLEEMTEAALQVLSRNPQGFYLFVEGGRIDHGHHEGKAYMALTDTVMFDNAIAKANELTSELDTLILATADHSHVFSFGGYTLRGTSIFGLAPSKASDNKSYTSILYGNGPGYVLGGGLRPDVNDSISEDPSYRQQAAVPLSSESHGGEDVAVFARGPQAHLVHGVQEETFVAHVMAFAGCVEPYTDCNLAPPAGTTDAAHPIEGRSVVPALLPLRAGTLLLLETATAP

SEQ ID NO: 12 - DNA, 1669 bp, synthetic construct
  misc_feature 1..1669: note = Synthetic polymer.
  source 1..1669: mol_type = other DNA
atgcaggggccctgggtgctgctgctgctgggcctgaggctacagctctccctgggcgtcatcccaggtaatgaggctccccaagctgttccacacacagggcaccccctcagccaggctgacctgatctctactctccccctggccagctgaggaggagaacccggccttctggaaccgccaggcagctgaggccctggatgctgccaagaagctgcagcccatccagaaggtcgccaagaacctcatcctcttcctgggcgatgggttgggggtgcccacggtgacagccaccaggatcctaaaggggcagaagaatggcaaactggggcctgagacgcccctggccatggaccgcttcccatacctggctctgtccaagacatacaatgtggacagacaggtgccagacagcgcagccacagccacggcctacctgtgcggggtcaaggccaacttccagaccatcggcttgagtgcagccgcccgctttaaccagtgcaacacgacacgcggcaatgaggtcatctccgtgatgaaccgggccaagcaagcaggaaagtcagtaggagtggtgaccaccacacgggtgcagcacgcctcgccagccggcacctacgcacacacagtgaaccgcaactggtactcagatgctgacatgcctgcctcagcccgccaggaggggtgccaggacatcgccactcagctcatctccaacatggacattgacgtgatccttggcggaggccgcaagtacatgtttcccatggggaccccagaccctgagtacccagctgatgccagccagaatggaatcaggctggacgggaagaacctggtgcaggaatggctggcaaagcaccagggtgcctggtatgtgtggaaccgcactgagctcatgcaggcgtccctggaccagtctgtgacccatctcatgggcctctttgagcccggagacacgaaatatgagatccaccgagaccccacactggacccctccctgatggagatgacagaggctgccctgcgcctgctgagcaggaacccccgcggcttctacctctttgtggagggcggccgcatcgaccatggtcatcatgagggtgtggcttaccaggcactcactgaggcggtcatgttcgacgacgccattgagagggcgggccagctcaccagcgaggaggacacgctgaccctcgtcaccgctgaccactcccatgtcttctcctttggtggctacaccttgcgagggagctccatcttcgggttggcccccagcaaggctcaggacagcaaagcctacacgtccatcctgtacggcaatggcccgggctacgtgttcaactcaggcgtgcgaccagacgtgaatgagagcgagagcgggagccccgattaccagcagcaggcggcggtgcccctgtcgtccgagacccacggaggcgaagacgtggcggtgtttgcgcgcggcccgcaggcgcacctggtgcatggtgtgcaggagcagagcttcgtagcgcatgtcatggccttcgctgcctgtctggagccctacacggcctgcgacctggcgcctcccgcctgcaccaccgacgccgcgcacccagttgccgcgtcgctgccactgctggccgggaccctgctgctgctgggggcgtccgctgctccctga

SEQ ID NO: 13 - DNA, 1671 bp, synthetic construct
  misc_feature 1..1671: note = Synthetic polymer.
  source 1..1671: mol_type = other DNA
atgcaggggccctgggtgctgctgctgctgggcctgaggctacagctctccctgggcgtcatcccagctgaggaggagaacccggccttctggaaccgccaggcagctgaggccctggatgctgccaagaagctgcagcccatccagaaggtcgccaagaacctcatcctcttcctgggcgatgggttgggggtgcccacggtgacagccaccaggatcctaaaggggcagaagaatggcaaactggggcctgagacgcccctggccatggaccgcttcccatacctggctctgtccaagacatacaatgtggacagacaggtgccagacagcgcagccacagccacggcctacctgtgcggggtcaaggccaacttccagaccatcggcttgagtgcagccgcccgctttaaccagtgcaacacgacacgcggcaatgaggtcatctccgtgatgaaccgggccaagcaagcaggaaagtcagtaggagtggtgaccaccacacgggtgcagcacgcctcgccagccggcacctacgcacacacagtgaaccgcaactggtactcagatgctgacatgcctgcctcagcccgccaggaggggtgccaggacatcgccactcagctcatctccaacatggacattgacgtgatccttggcggaggccgcaagtacatgtttcccatggggaccccagaccctgagtacccagctgatgccagccagaatggaatcaggctggacgggaagaacctggtgcaggaatggctggcaaagcaccagggtgcctggtatgtgtggaaccgcactgagctcatgcaggcgtccctggaccagtctgtgacccatctcatgggcctctttgagcccggagacacgaaatatgagatccaccgagaccccacactggacccctccctgatggagatgacagaggctgccctgcgcctgctgagcaggaacccccgcggcttctacctctttgtggagggcggccgcatcgaccatggtcatcatgagggtgtggcttaccaggcactcactgaggcggtcatgttcgacgacgccattgagagggcgggccagctcaccagcgaggaggacacgctgaccctcgtcaccgctgaccactcccatgtcttctcctttggtggctacaccttgcgagggagctccatcttcgggttggcccccagcaaggctcaggacagcaaagcctacacgtccatcctgtacggcaatggcccgggctacgtgttcaactcaggcgtgcgaccagacgtgaatgagagcgagagcgggagccccgattaccagcagcaggcggcggtgcccctgtcgtccgagacccacggaggcgaagacgtggcggtgtttgcgcgcggcccgcaggcgcacctggtgcatggtgtgcaggagcagagcttcgtagcgcatgtcatggccttcgctgcctgtctggagccctacacggcctgcgacctggcgcctcccgcctgcaccaccgacgccgcgcacccagttgccgcgtcgctgccactgctggccgggaccctgctgctgctgggggcgtccgctgctccctgatttactaaaaccttgaaataaaattgtaaaacatcagtttgaaggcctgactctcagggtagttcttttttaattctgggtttt

SEQ ID NO: 14 - DNA, 1672 bp, synthetic construct
  misc_feature 1..1672: note = Synthetic polymer.
  source 1..1672: mol_type = other DNA
atgcagtgggcctgtgtgctgctgctgctgggcctgtggctacagctctccctcaccttcatcccaggtaatcaggcggctcccagcagcccctactcacaggggcggctctaggctgacctgaccaacactctccccttgggcagctgaggaggaagaccccgccttctggaaccgccaggcagcccaggcccttgatgtagccaagaagttgcagccgatccagacagctgccaagaatgtcatcctcttcttgggggatgggatgggggtgcctacggtgacagccactcggatcctaaaggggcagatgaatggtaagctgggacctgagacacccctggccatggaccagttcccatacgtggctctgtccaagacatacaacgtggacagacaggtgccagacagcgcaggcactgccactgcctacctgtgtggggtcaagggcaactacaaaaccattggtgtaagtgcagccgcccgctacaaccagtgcaacacaacaagtggcaatgaggtcacgtctgtgatgaaccgggccaagaaagcaggaaagtcagtgggagtggtgaccacctccagggtgcagcatgcctccccagccggtgcttatgcacacacggtgaaccgaaactggtactcagatgccgacctgcctgccgatgcacagacgtatggctgccaggacatcgccacacaactggtcaacaacatggatattgacgtgatcctgggtggaggccgaatgtacatgtttcctgaggggaccccggatcctgaatacccatacgatgtcaatcagactggagtccggaaggacaagcggaatctggtgcaggagtggcaggccaagcaccagggagcccagtatgtgtggaaccgcacggagctccttcaggcagccaatgaccccagtgtaacacacctcatgggcctctttgagccggcagacatgaagtataatgttcagcaagaccccaccaaggacccgaccctggaggagatgacggaggcggccctgcaagtgctgagcaggaacccccagggcttctacctcttcgtggagggaggccgcattgaccacggtcaccatgaaggcaaagcttatatggcactgactgatacagtcatgtttgacaatgccatcgccaaggctaacgagctcactagcgaactggacacgctgatccttgccactgcagaccactcccatgtcttctcttttggtggctacacactgcgtgggacctccattttcggtctggcccccagcaaggcctcagacaacaagtcctacacctccatcctctatggcaatggccctggctacgtgcttggtgggggcttaaggcccgatgttaatgacagcataagcgaggacccctcgtaccggcagcaggcggccgtgcccctgtctagtgagtcccacgggggcgaggacgtggcggtgttcgcgcgaggcccgcaggcgcacctggtgcacggcgtgcaggaggagaccttcgtggcgcacgtcatggcctttgcgggctgcgtggagccctacaccgactgcaatctgccggccccctctggcctctccgacgccgcgcacctggcggccagcccgccttcgctggcgctgctggccggggcgatgctgctgctgctggcgcctgccttgtactga

SEQ ID NO: 15 - DNA, 1869 bp, synthetic construct
  misc_feature 1..1869: note = Synthetic polymer.
  source 1..1869: mol_type = other DNA
atgcagtgggcctgtgtgctgctgctgctgggcctgtggctacagctctccctcaccttcatcccagctgaggaggaagaccccgccttctggaaccgccaggcagcccaggcccttgatgtagccaagaagttgcagccgatccagacagctgccaagaatgtcatcctcttcttgggggatgggatgggggtgcctacggtgacagccactcggatcctaaaggggcagatgaatggtaagctgggacctgagacacccctggccatggaccagttcccatacgtggctctgtccaagacatacaacgtggacagacaggtgccagacagcgcaggcactgccactgcctacctgtgtggggtcaagggcaactacaaaaccattggtgtaagtgcagccgcccgctacaaccagtgcaacacaacaagtggcaatgaggtcacgtctgtgatgaaccgggccaagaaagcaggaaagtcagtgggagtggtgaccacctccagggtgcagcatgcctccccagccggtgcttatgcacacacggtgaaccgaaactggtactcagatgccgacctgcctgccgatgcacagacgtatggctgccaggacatcgccacacaactggtcaacaacatggatattgacgtgatcctgggtggaggccgaatgtacatgtttcctgaggggaccccggatcctgaatacccatacgatgtcaatcagactggagtccggaaggacaagcggaatctggtgcaggagtggcaggccaagcaccagggagcccagtatgtgtggaaccgcacggagctccttcaggcagccaatgaccccagtgtaacacacctcatgggcctctttgagccggcagacatgaagtataatgttcagcaagaccccaccaaggacccgaccctggaggagatgacggaggcggccctgcaagtgctgagcaggaacccccagggcttctacctcttcgtggagggaggccgcattgaccacggtcaccatgaaggcaaagcttatatggcactgactgatacagtcatgtttgacaatgccatcgccaaggctaacgagctcactagcgaactggacacgctgatccttgccactgcagaccactcccatgtcttctcttttggtggctacacactgcgtgggacctccattttcggtctggcccccagcaaggcctcagacaacaagtcctacacctccatcctctatggcaatggccctggctacgtgcttggtgggggcttaaggcccgatgttaatgacagcataagcgaggacccctcgtaccggcagcaggcggccgtgcccctgtctagtgagtcccacgggggcgaggacgtggcggtgttcgcgcgaggcccgcaggcgcacctggtgcacggcgtgcaggaggagaccttcgtggcgcacgtcatggcctttgcgggctgcgtggagccctacaccgactgcaatctgccggccccctctggcctctccgacgccgcgcacctggcggccagcccgccttcgctggcgctgctggccggggcgatgctgctgctgctggcgcctgccttgtactgaggggacccgggggtggggacacaggccccgccctccctgggaggcaggaagcagctctcaaataaactgttctaagtatgatacaggagtgatacatgtgtgaagagaagcccttaggtgggggcacagagtgtctgggtgaggggggtcagggtcacatcaggaggttagggaggggttgatgaagggctgacgttgagcaaagaccaaaggcaactcagaaggacagtggtgcaggactgggtgtggtcagcagggggactggttgggggatcc

SEQ ID NO: 16 - DNA, 1260 bp, synthetic construct
  misc_feature 1..1260: note = Synthetic polymer.
  source 1..1260: mol_type = other DNA
aaaaaacaagacaaagctgagatcagaaatgtcattgtgatgataggcgacggcatggggacgccttacataagagcctaccgttccatgaaaaataacggtgacacaccgaataacccgaagttaacagaatttgaccggaacctgacaggcatgatgatgacgcatccggatgaccctgactataatattacagattcagcagcagccggaacagcattagcgacaggcgttaagacatataacaatgcaattggcgtcgataaaaacggaaaaaaagtgaaatctgtacttgaagaggccaaacagcaaggcaagtcaacagggcttgtcgccacgtctgaaattaaccacgccactccagccgcatatggcgcccacaatgaatcacggaaaaacatggaccaaatcgccaacagctatatggatgacaagataaaaggcaaacataaaatagacgtgctgctcggcggcggaaaatcttattttaaccgcaagaacagaaacttgacaaaggaattcaaacaagccggctacagctatgtgacaactaaacaagcattgaaaaaaaataaagatcagcaggtgctcgggcttttcgcagatggagggcttgctaaagcgctcgaccgtgacagtaaaacaccgtctctcaaagacatgacggtttcagcaattgatcgcctgaaccaaaataaaaaaggatttttcttgatggtcgaagggagccagattgactgggcggcccatgacaatgatacagtaggagccatgagcgaggttaaagattttgaacaggcctataaagccgcgattgaatttgcgaaaaaagacaaacatacacttgtgattgcaactgctgaccatacaaccggcggctttaccattggcgcaaacggggaaaagaattggcacgcagaaccgattctctccgctaagaaaacacctgaattcatggccaaaaaaatcagtgaaggcaagccggttaaagatgtgctcgcccgctatgccaatctgaaagtcacatctgaagaaatcaaaagcgttgaagcagctgcacaggctgacaaaagcaaaggggcctccaaagccatcatcaagatttttaatacccgctccaacagcggatggacgagtaccgatcataccggcgaagaagtaccggtatacgcgtacggccccggaaaagaaaaattccgcggattgattaacaatacggaccaggcaaacatcatatttaagattttaaaaactggaaaa

SEQ ID NO: 17 - protein, 420 aa, synthetic construct
  REGION 1..420: note = Synthetic polymer.
KKQDKAEIRNVIVMIGDGMGTPYIRAYRSMKNNGDTPNNPKLTEFDRNLTGMMMTHPDDPDYNITDSAAAGTALATGVKTYNNAIGVDKNGKKVKSVLEEAKQQGKSTGLVATSEINHATPAAYGAHNESRKNMDQIANSYMDDKIKGKHKIDVLLGGGKSYFNRKNRNLTKEFKQAGYSYVTTKQALKKNKDQQVLGLFADGGLAKALDRDSKTPSLKDMTVSAIDRLNQNKKGFFLMVEGSQIDWAAHDNDTVGAMSEVKDFEQAYKAAIEFAKKDKHTLVIATADHTTGGFTIGANGEKNWHAEPILSAKKTPEFMAKKISEGKPVKDVLARYANLKVTSEEIKSVEAAAQADKSKGASKAIIKIFNTRSNSGWTSTDHTGEEVPVYAYGPGKEKFRGLINNTDQANIIFKILKTGK

SEQ ID NO: 18 - protein, 776 aa, synthetic construct
  REGION 1..776: note = Synthetic polymer.
MQGPWVLLLLGLRLQLSLGVIPAEEENPAFWNRQAAEALDAAKKLQPIQKVAKNLILFLGDGLGVPTVTATRILKGQKNGKLGPETPLAMDRFPYLALSKTYNVDRQVPDSAATATAYLCGVKANFQTIGLSAAARFNQCNTTRGNEVISVMNRAKQAGKSVGVVTTTRVQHASPAGTYAHTVNRNWYSDADMPASARQEGCQDIATQLISNMDIDVILGGGRKYMFPMGTPDPEYPADASQNGIRLDGKNLVQEWLAKHQGAWYVWNRTELMQASLDQSVTHLMGLFEPGDTKYEIHRDPTLDPSLMEMTEAALRLLSRNPRGFYLFVEGGRIDHGHHEGVAYQALTEAVMFDDAIERAGQLTSEEDTLTLVTADHSHVFSFGGYTLRGSSIFGLAPSKAQDSKAYTSILYGNGPGYVFNSGVRPDVNESESGSPDYQQQAAVPLSSETHGGEDVAVFARGPQAHLVHGVQEQSFVAHVMAFAACLEPYTACDLAPPACTTDAAHPVAASLPLLAGTLLLLGASAAPGGSGGSGGGGSGGGGSEPKSCDKTHTCPPCPAPELLGGPSVFLFPPKPKDTLMISRTPEVTCVVVDVSHEDPQVKFNWYVDGVQVHNAKTKPREQQYNSTYRVVSVLTVLHQNWLDGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 16 aa, synthetic construct
  REGION 1..16: note = Synthetic polymer.
GGSGGSGGGGSGGGGS

SEQ ID NO: 20 - protein, 5 aa, synthetic construct
  REGION 1..5: note = Synthetic polymer.
GGGGS

SEQ ID NO: 21 - protein, 10 aa, synthetic construct
  REGION 1..10: note = Synthetic polymer.
GGGGSGGGGS

SEQ ID NO: 22 - protein, 15 aa, synthetic construct
  REGION 1..15: note = Synthetic polymer.
GGGGSGGGGSGGGGS

SEQ ID NO: 23 - protein, 20 aa, synthetic construct
  REGION 1..20: note = Synthetic polymer.
GGGGSGGGGSGGGGSGGGGS

SEQ ID NO: 24 - protein, 8 aa, synthetic construct
  REGION 1..8: note = Synthetic polymer.
GGGGGGGG

SEQ ID NO: 25 - protein, 6 aa, synthetic construct
  REGION 1..6: note = Synthetic polymer.
GGGGGG

SEQ ID NO: 26 - protein, 5 aa, synthetic construct
  REGION 1..5: note = Synthetic polymer.
EAAAK

SEQ ID NO: 27 - protein, 10 aa, synthetic construct
  REGION 1..10: note = Synthetic polymer.
EAAAKEAAAK

SEQ ID NO: 28 - protein, 15 aa, synthetic construct
  REGION 1..15: note = Synthetic polymer.
EAAAKEAAAKEAAAK

SEQ ID NO: 29 - protein, 12 aa, synthetic construct
  REGION 1..12: note = Synthetic polymer.
AEAAAKEAAAKA

SEQ ID NO: 30 - protein, 17 aa, synthetic construct
  REGION 1..17: note = Synthetic polymer.
AEAAAKEAAAKEAAAKA

SEQ ID NO: 31 - protein, 22 aa, synthetic construct
  REGION 1..22: note = Synthetic polymer.
AEAAAKEAAAKEAAAKEAAAKA

SEQ ID NO: 32 - protein, 27 aa, synthetic construct
  REGION 1..27: note = Synthetic polymer.
AEAAAKEAAAKEAAAKEAAAKEAAAKA

SEQ ID NO: 33 - protein, 46 aa, synthetic construct
  REGION 1..46: note = Synthetic polymer.
AEAAAKEAAAKEAAAKEAAAKALEAEAAAKEAAAKEAAAKEAAAKA

SEQ ID NO: 34 - protein, 5 aa, synthetic construct
  REGION 1..5: note = Synthetic polymer.
PAPAP

SEQ ID NO: 35 - protein, 18 aa, synthetic construct
  REGION 1..18: note = Synthetic polymer.
KESGSVSSEQLAQFRSLD

SEQ ID NO: 36 - protein, 13 aa, synthetic construct
  REGION 1..13: note = Synthetic polymer.
EGKSSGSGSESKS

SEQ ID NO: 37 - protein, 12 aa, synthetic construct
  REGION 1..12: note = Synthetic polymer.
GSAGSAAGSGEF

SEQ ID NO: 38 - DNA, 13 bp, synthetic construct
  misc_feature 1..13: note = Synthetic polymer.
  source 1..13: mol_type = other DNA
gccgccaccatgg

SEQ ID NO: 39-40 - skipped (2 entries)

SEQ ID NO: 41 - protein, 487 aa, synthetic construct
  REGION 1..487: note = Synthetic polymer.
LIPAEEENPAFWNRQAAQALDVAKKLQPIQTAAKNVILFLGDGMGVPTVTATRILKGQMNGKLGPETPLAMDQFPYVALSKTYNVDRQVPDSAGTATAYLCGVKGNYRTIGVSAAARYNQCNTTRGNEVTSVINRAKKAGKAVGVVTTTRVQHASPAGAYAHTVNRNWYSDADLPADAQKNGCQDIAAQLVYNMDIDVILGGGRMYMFPEGTPDPEYPDDASVNGVRKDKQNLVQEWQAKHQGAQYVWNRTALLQAADDSSVTHLMGLFEPADMKYNVQQDHTKDPTLAEMTEAALQVLSRNPRGFYLFVEGGRIDHGHHDGKAYMALTEAIMFDNAIAKANELTSELDTLILVTADHSHVFSFGGYTLRGTSIFGLAPGKALDSKSYTSILYGNGPGYALGGGSRPDVNGSTSEEPSYRQQAAVPLASETHGGEDVAVFARGPQAHLVHGVQEETFVAHIMAFAGCVEPYTDCNLPAPATATSIPD